(19) United States
(12) Patent Application Publication
(10) Pub. No.: US 2026/0193617 A1
(43) Pub. Date: Jul. 9, 2026

(54) SHAPED ORGANOID COMPOSITIONS AND METHODS OF MAKING SAME

(71) Applicant: CHILDREN'S HOSPITAL MEDICAL CENTER, Cincinnati, OH (US)

(72) Inventors: Michael HELMRATH, Cincinnati, OH (US); Maxime MAHE, Reze (FR); Holly POLING, Cincinnati, OH (US)

(21) Appl. No.: 19/427,030

(22) Filed: Dec. 19, 2025

Related U.S. Application Data
(63) Continuation of application No. 17/595,494, filed on May 29, 2020, now Pat. No. 12,534,709.
(60) Provisional application No. 62/855,557, filed on May 31, 2019.
(60) Provisional application No. 62/909,868, filed on Oct. 3, 2019.
(60) Provisional application No. 62/958,367, filed on Jan. 8, 2020.

(51) Int. Cl. C12N 5/071; A61K 35/38; A61P 1/00; C12M 3/00; C12N 5/00; C12N 5/077; C12N 5/0793

(52) U.S. Cl. CPC C12N 5/0697; A61K 35/38; A61P 1/00; C12M 21/08; C12N 5/0062; C12N 5/0619; C12N 5/0656; C12N 5/0679; C12N 2506/45; C12N 2513/00; C12N 2527/00; C12N 2535/00

(57) ABSTRACT

Disclosed herein are organoid compositions that are manipulated to form shaped or elongated morphologies that more closely resemble native organ structures. These shaped organoids are advantageous for purposes such as studying organellar organization and for transplants compared to unformed organoids. Also disclosed herein are methods of producing said shaped or elongated organoid compositions.

### CROSS-REFERENCE TO RELATED APPLICATIONS

This application is a Continuation of U.S. Non-Provisional application Ser. No. 17/595,494, filed Nov. 17, 2021, which is a 371 National Stage Application from PCT/US2020/035411 filed on May 29, 2020 and claims the benefit of priority of U.S. Provisional Patent Application No. 62/855,557, filed May 31, 2019, U.S. Provisional Patent Application No. 62/909,868, filed Oct. 3, 2019, and U.S. Provisional Patent Application No. 62/958,367, filed Jan. 8, 2020, each of which is hereby expressly incorporated by reference in its entirety.

### STATEMENT REGARDING FEDERALLY SPONSORED R&D

This invention was made with government support under U01DK103117 awarded by the National Institutes of Health. The government has certain rights to the invention.

### FIELD OF THE INVENTION

Aspects of the present disclosure relate generally to organoid compositions and methods of making said organoid compositions. The organoids disclosed herein have shaped or elongated structures that more closely resemble in vivo organ tissue.

### BACKGROUND

Existing methods for providing organoids, such as intestinal organoids, are limited in their ability to form structures well-suited for transplant with subsequent functional implementation. Specifically, current methods of obtaining organoids derived from pluripotent stem cells, particularly induced pluripotent stem cells, result in organoids having a spherical structure, which does not naturally elongate when implanted, thus failing to supply a structure having a configuration similar to that of native structures. In addition, the presence of axial force may influence the development of the organoid tissue. While existing gastrointestinal organoids comprise a lumen, the limitation in shape and size available using existing methods is of limited utility for clinical implementation. Thus, there is a present need for organoid tissue grown in vitro, derived from a patient such as a human patient, which has improved suitability for transplant and improved function following transplant.

### SUMMARY

Some aspects of the present disclosure relate generally to methods of producing a shaped gastrointestinal organoid. In some embodiments, the gastrointestinal organoid comprises a lumen. In some embodiments, the methods comprise placing a plurality of spheroids into a collection channel comprising a predetermined shape, and culturing the plurality of spheroids in the collection channel to differentiate the plurality of spheroids into the shaped gastrointestinal organoid having the predetermined shape. In some embodiments, the shaped gastrointestinal organoid comprises a condensed mesenchyme and lumen. In some embodiments, the collection channel has a non-spherical shape and the shaped gastrointestinal organoid is non-spherical gastrointestinal organoid. In some embodiments, the collection channel has an elongated shape and the shaped gastrointestinal organoid is an elongated gastrointestinal organoid. In some embodiments, the elongated gastrointestinal organoid comprises an elongate length and a diameter. In some embodiments, the elongate length is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 millimeters, or any length within a range defined by any two of the aforementioned lengths, for example, 1 to 50 mm, 10 to 40 mm, 20 to 30 mm, 1 to 30 mm, or 20 to 50 mm. In some embodiments, the diameter is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any diameter within a range defined by any two of the aforementioned diameters, for example, 0.2 μm to 3000 μm, 200 μm to 1500 μm, 500 μm to 1000 μm, 0.2 μm to 1000 μm, or 500 μm to 3000 μm. In some embodiments, the ratio of the elongate length to the diameter is, is about, is at least, is at least about, is not more than, or is not more than about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 500000, 60000, 70000, 80000, 90000, 100000, 200000, 300000, 400000, or 500000, or any ratio between a range defined by any two of the aforementioned ratios, for example, 1 to 500000, 100 to 500000, 1000 to 10000, 1 to 500000, or 1000 to 500000. In some embodiments, the lumen is not continuous throughout the elongate length of the shaped gastrointestinal organoid. In some embodiments, the shaped gastrointestinal organoid is a shaped human gastrointestinal organoid. In some embodiments, the plurality of spheroids are cultured in the collection channel for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the plurality of spheroids fuse at the mesenchyme of the plurality of spheroids. In some embodiments, the shaped gastrointestinal organoid undergoes spontaneous innervation. In some embodiments, the shaped gastrointestinal organoid further comprises enteric neuronal cells or enteric neuronal progenitor cells. In some embodiments, the shaped gastrointestinal organoid further comprises one or more myenteric plexuses comprising cells that express the neuronal marker PGP9.5. In some embodiments, the shaped gastrointestinal organoid has neuronal activity. In some embodiments, the methods comprise inducing a mechanical strain on the shaped gastrointestinal organoid, wherein the mechanical strain promotes the spontaneous innervation of the shaped gastrointestinal organoid, or decreases maturation time of the shaped gastrointestinal organoid, or both. In some embodiments, the mechanical strain is a uniaxial tensile strain. In some embodiments, the shaped gastrointestinal organoid further comprises a polarized, columnar epithelium surrounded by mesenchyme, wherein the mesenchyme comprises a smooth muscle-like layer. In some embodiments, the shaped gastrointestinal organoid further comprises an epithelium patterned into crypt-like proliferative zones or villus-like structures, or both. In some embodiments, the shaped gastrointestinal organoid further comprises laminated longitudinal and circular muscle. In some embodiments, the shaped gastrointestinal organoid further comprises markers of smooth muscle or intestinal sub-epithelial myofibroblast cells, or both. In some embodiments, the shaped gastrointestinal organoid further comprises one or more of enterocytes, enteroendocrine cells, goblet cells, Paneth cells, or any combination thereof. In some embodiments, the shaped gastrointestinal organoid further comprises cells that express one or more of villin, Muc2, DEFA5, CHGA, or OLFM4, or any combination thereof. In some embodiments, the shaped gastrointestinal organoid is derived from induced pluripotent stem cells reprogrammed from PBMC cells, a biopsy tissue sample, or Sendai virus-transduced somatic cells. In some embodiments, the shaped gastrointestinal organoid is vascularized in vitro. In some embodiments, the shaped gastrointestinal organoid is vascularized upon engraftment into an individual. In some embodiments, the plurality of spheroids is a plurality of mid-hindgut spheroids and the shaped gastrointestinal organoid is a shaped intestinal organoid. In some embodiments, the plurality of spheroids is a plurality of hindgut spheroids and the shaped gastrointestinal organoid is a shaped colonic organoid. In some embodiments, the plurality of spheroids is a plurality of anterior foregut spheroids and the shaped gastrointestinal organoid is an esophageal organoid. In some embodiments, the plurality of spheroids is a plurality of posterior foregut spheroids and the shaped gastrointestinal organoid is a gastric organoid. In some embodiments, the methods comprise culturing induced pluripotent stem cells under conditions sufficient to differentiate the induced pluripotent stem cells into definitive endoderm, culturing the definitive endoderm under conditions sufficient to differentiate the definitive endoderm into the plurality of spheroids, and collecting the plurality of spheroids prior to placing the plurality of spheroids into the collection channel. In some embodiments, the collecting step comprises contacting the plurality of spheroids with a binding material capable of binding to the plurality of spheroids. In some embodiments, the binding material is selected from one or more of a wire, a string, and a fiber. In some embodiments, the plurality of spheroids is contacted with a scaffold strand.

Some aspects of the present disclosure relate generally to methods of treating an individual having compromised gastrointestinal function. In some embodiments, the methods comprise transplanting a gastrointestinal organoid into the individual. In some embodiments, the gastrointestinal organoid is any one of the shaped gastrointestinal organoids described herein. In some embodiments, the gastrointestinal organoid is autologous or allogeneic to the individual. In some embodiments, the gastrointestinal organoid is prepared from induced pluripotent stem cells obtained from the individual. In some embodiments, the individual is in need of a gastrointestinal transplant. In some embodiments, the gastrointestinal function is intestinal function and the gastrointestinal organoid is an intestinal organoid. In some embodiments, the gastrointestinal function is colonic function and the gastrointestinal organoid is a colonic organoid. In some embodiments, the gastrointestinal function is esophageal function and the gastrointestinal organoid is an esophageal organoid. In some embodiments, the gastrointestinal function is stomach function and the gastrointestinal organoid is a gastric organoid.

Some aspects of the present disclosure relate generally to a formation tray for culturing one or more shaped gastrointestinal organoids. In some embodiments, the formation tray comprises one or more collection channels configured to receive one or more plurality of spheroids therein. In some embodiments, the one or more collection channels have a predetermined shape and are configured to gather the one or more plurality of spheroids together such that the one or more plurality of spheroids collect into the predetermined shape and wherein the one or more plurality of spheroids differentiate into the one or more shaped gastrointestinal organoids having the predetermined shape. In some embodiments, the one or more collection channels are made of a biocompatible material configured to inhibit the one or more plurality of spheroids from attaching thereto. In some embodiments, the one or more collection channels further comprise one or more plurality of spheroids positioned therein. In some embodiments, the one or more collection channels further comprise a cell culture media or extracellular matrix, or both, therein. In some embodiments, the one or more collection channels further comprise the one or more gastrointestinal organoids positioned therein. In some embodiments, the one or more gastrointestinal organoids is any one or more shaped gastrointestinal organoids described herein.

Some aspects of the present disclosure relate generally to a kit for culturing a gastrointestinal organoid. In some embodiments, the kit comprises a formation tray comprising one or more collection channels. In some embodiments, the formation tray is any one of the formation trays described herein. In some embodiments, the kit comprises a plurality of spheroids configured to be received within the one or more collection channels. In some embodiments, the kit comprises a cell culture media configured to be received within the one or more collection channels.

Embodiments of the present invention provided herein are described by way of the following numbered alternatives:

1. A method of obtaining an elongated human intestinal organoid comprising a lumen, comprising:
(a) culturing a source of induced pluripotent stem cells under conditions sufficient to form definitive endoderm;
(b) culturing said definitive endoderm until a plurality of spheroids is formed;
(c) collecting said plurality of spheroids;
(d) placing said plurality of spheroids in a collection channel; and
(e) forming a human intestinal organoid comprising a condensed mesenchyme and lumen from said plurality of spheroids in said collection channel;
wherein said collection channel has at least one region that is at least partially tubular in structure.

2. The method of alternative 1, wherein said collecting comprises contacting said spheroids with a binding material capable of binding to said spheroids.

3. The method of alternative 2, wherein said binding material is selected from one or more of a wire, a string, and a fiber.

4. The method of any preceding alternative, wherein said collection channel has an elongated shape.

5. The method of any preceding alternative, wherein said collection channel has a length of at least 1 cm, or at least 2 cm, or at least 3 cm, or at least 4 cm, or at least 5 cm, or from about 1 cm to about 100 cm.

6. The method of any preceding alternative, wherein said method is carried out in a device having a scaffold strand.

7. The method of any preceding alternative, wherein said plurality of spheroids are in said collection channel for a period of 1 day to 20 days, or 2 days to 18 days, or 3 days to 17 days, or 4 days to 16 days, or 5 days to 15 days, or 6 days to 14 days.

8. The method of any preceding alternative, wherein said plurality of spheroids fuse at the mesenchyme of said spheroids.

9. The method of alternative 1, wherein said elongated intestinal organoid is transplanted into a host at about day 14, or from about day 13 to day 15, or from about day 12 to about day 16, or about day 11 to about day 17, preferably wherein said elongated intestinal organoid is transplanted adjacent to a bowel of said host.

10. The method of any preceding alternative, wherein said intestinal organoid forms a blood supply in vitro.

11. The method of any preceding alternative, wherein said intestinal organoid forms a blood supply after engraftment into an individual.

12. The method of alternative 1, further comprising the step of transplanting said elongated intestinal organoid into a host, wherein said host is selected from an immunodeficient mammal and an individual in need of said transplanting step.

13. The method of any preceding alternative, wherein said spheroid is a mid/hindgut spheroid.

14. A tray for culturing an intestinal organoid, comprising:
a base formed from a biocompatible material configured to inhibit a plurality of spheroids from attaching thereto; and
a collection channel extending through the base and configured to receive the plurality of spheroids therein, wherein the collection channel is elongated and configured to gather the plurality of spheroids together such that the plurality of spheroids define a predetermined shape for culturing the plurality of spheroids to the intestinal organoid having the predetermined shape.

15. The tray of alternative 14, further comprising a culture media positioned within the collection channel.

16. The tray of alternative 15, further comprising a plurality of spheroids positioned within the collection channel.

17. A kit for culturing an intestinal organoid, comprising:
a collection channel; and
a culture media configured to be received within the collection channel.

18. The kit of alternative 17, further comprising a plurality of spheroids configured to be received within the collection channel with the culture media.

19. A method of treating an individual having compromised intestinal function, comprising transplanting into said individual an organoid derived from an induced pluripotent stem cell, wherein said organoid comprises mesenchymal tissue and endodermal tissue.

20. The method of alternative 19, wherein said organoid further comprises neuronal tissues.

21. The method of alternative 19, wherein said induced pluripotent stem cell is derived from said individual.

22. The method of alternative 19, wherein said individual is in need of an intestinal transplant.

23. The method of any of alternatives 19 to 22, wherein said organoids are derived from induced pluripotent stem cells generated from Sendai virus transduced somatic cells.

24. The method of any of alternatives 19 to 23, wherein said organoids comprise a polarized, columnar epithelium surrounded by mesenchyme that includes a smooth muscle-like layer.

25. The method of any of alternatives 19 to 24, wherein said organoids comprise an epithelium patterned into crypt-like proliferative zones and villus-like structures.

26. The method of any of alternatives 19 to 25, wherein said organoids comprise laminated, longitudinal, and circular muscle.

27. The method of any of alternatives 19 to 26, wherein said organoids comprise markers of smooth muscle and intestinal sub-epithelial myofibroblast cells.

28. The method of any of alternatives 19 to 27, wherein said organoids comprise enterocytes, goblet, Paneth, and enteroendocrine cells or secretory, endocrine and absorptive cell types.

29. The method of any of alternatives 19 to 28, wherein said organoids have neuronal activity.

30. The method of any of alternatives 19 to 29, further comprising the step of applying tension to said organoids to decrease maturation time.

### BRIEF DESCRIPTION OF THE DRAWINGS

In addition to the features described above, additional features and variations will be readily apparent from the following descriptions of the drawings and exemplary embodiments. It is to be understood that these drawings depict embodiments and are not intended to be limiting in scope.

FIG. 1A depicts an embodiment of the clinical progression from intestinal failure to intestinal transplantation.

FIG. 1B depicts an embodiment of the number of individuals on the transplant waiting list in the United States from 1991 to 2017.

FIG. 2A depicts an embodiment of a perspective view of an example formation tray having a plurality of collection channels for gather a plurality of spheroids.

FIG. 2B depicts an embodiment of a cross-sectional view of the example formation tray of FIG. 2A taken along section line 2-2 of FIG. 2A.

FIG. 2C depicts an embodiment of a cross-sectional view of the example formation tray of FIG. 2A taken along section line 3-3 of FIG. 2A.

FIG. 3A depicts an embodiment of a schematic perspective view of a biocompatible container culturing a plurality of pluripotent stem cells.

FIGS. 3B-D depict embodiments of a schematic perspective view of the biocompatible container of FIG. 4 but (FIG. 3B) showing the plurality of pluripotent stem cells cultured to a definitive endoderm, (FIG. 3C) showing the pluripotent stem cells cultured to a plurality of spheroids from a definitive endoderm intermediate, or (FIG. 3D) showing the plurality of pluripotent stem cells cultured to a plurality of spheroids arranged according to a predetermined arrangement induced by attraction to a scaffold thread.

FIG. 4 depicts an embodiment of an enlarged top view of the formation tray of FIG. 2A with a plurality of spheroids within the plurality of collection channels cultured therein during day 1 (d1), day 3 (d3), or day 5 (d5).

FIG. 5 depicts an embodiment of an elongate intestinal organoid (46) implanted into a host organism (44).

FIG. 6A depicts an embodiment of a light micrograph of an unshaped HIO in culture. Also shown is an embodiment of hematoxylin and eosin stained sections of day 14 (panel A) and day 28 (panel B) unshaped HIOs. Scale bar=0.5 mm.

FIG. 6B depicts an embodiment of hematoxylin and eosin stained sections of tHIO harvested 2, 4, and 8 weeks post-transplantation (top) compared to historical sections of fetal human intestine (bottom). Development of epithelia structuration in tHIOs progresses in a similar fashion to native tissue. GA: gestational age, Human data reproduced from Grand et al. (1976).

FIG. 7A depicts an embodiment of a methodology of producing the elongated HIOs and subsequent transplantation into a host organism.

FIG. 7B depicts an embodiment of hematoxylin and eosin stained sections of day 6 (panel A), day 14 (panel B), and day 28 (panel C) in vitro shaped elongated HIOs (g-HIO) structures. Scale bar=1 mm. Note: structure in panel B is not full length.

FIG. 8A depicts an embodiment of the formation of elongated HIOs and successful generation of a human PSC-derived tubular intestinal organoid with continuous epithelium. Panel A shows an image of an ABS mold and PDMS scaffold formation trays. Panel B shows a scanning electron micrograph of the cross section (top) and end (bottom) of the PDMS scaffold formation tray. Panel C shows in vitro images of spheroids in grooves at 1, 3, and 5 days of culture. Panel D shows an operative image of a day 14 organoid structure at the time of transplantation. Panel E shows a gross image of engrafted day 14 organoid structure after six weeks of transplantation. The dashed line indicates plane of dissection. Panel F shows a hematoxylin and eosin stained section of the graft of panel E. Panel G shows a tile scan of the hematoxylin and eosin stained section of the graft. An area of adjacent mouse tissue is labeled. Continuous epithelium across the whole tissue is observed.

FIG. 8B depicts an embodiment of a transplantation of an elongated intestinal organoid and resulting vascularization. Also shown is histology of the elongated intestinal organoid after successful engraftment.

FIG. 8C depicts an embodiment of images of transplanted g-HIOs at the time of harvest. Top panels show whole day 14 g-HIOs after transplantation in the mesentery of immunocompromised rats. Bottom panel shows whole day 28 unshaped HIO after 8 weeks of transplantation in the mesentery of an immunocompromised rat. Unshaped HIOs made using the conventional protocol did not engraft into immunocompromised rats when transplanted at day 14.

FIGS. 9A-B depict an embodiment of HIOs which grow significantly upon in vivo transplantation. Transplanted HIOs (tHIOs) are significantly larger than in vitro HIOs at the time of harvest.

FIG. 9C depicts an embodiment of transplanted HIOs that resemble human intestine. tHIOs comprise major intestinal cell lineages including mesenchyme, enterocytes (VIL1), enteroendocrine cells (CHGA), goblet cells (MUC2), and Paneth cells (DEFA5). In addition, they stain positive for a marker of stem cell activity (OLFM4).

FIG. 9D depicts an embodiment of organoid-to-intestine anastomoses in a mesentery transplantation model. 50% of mice survived to 21 days at the time of harvest.

FIG. 9E depicts an embodiment of the spontaneous development of myenteric plexuses that occur post-transplantation in tHIOs prepared in grooves (g-tHIOs). Histology of g-tHIOs reveal a robust network of myenteric plexuses throughout the harvested tissue (panel A, left). At higher magnification, bundle structures are visible (panel A, right). Immunofluorescence staining for a pan-neuronal marker (PGP9.5) and a human specific marker (KU80). Colocalization of these proteins demonstrate human origin of the neuronal components (panel B). Immunohistochemical staining for pan-neuronal marker PGP9.5 in tHIO combined with neural crest cells to form an enteric nervous system (tHIO+ENS), g-tHIO, and adult human small intestine (panel C). Quantification of myenteric plexuses (PGP9.5+cell bundles) from panel C (panel D). Plexus size in g-tHIOs is significantly larger when compared to tHIO+ENS using the traditional differentiation protocol. All scale bars=100 μm.

FIG. 10A depicts an embodiment of transcriptomic segregation of in vitro g-HIOs and unshaped HIOs. Principal component analysis of spheroids, day 28 unshaped HIOs and day 28 g-HIOs (panel A). Heatmap of day 28 unshaped HIOs and g-HIOs (panel B). Venn diagram of differentially expressed genes between day 28 unshaped HIOs and g-HIOs (panel C). List of top ten biological processes enriched in g-HIOs relate to neuronal development (panel D).

FIG. 10B depicts an embodiment of transcriptomic profiles of g-HIOs developing in vitro. Principal component analysis of spheroids, day 6 g-HIO, day 14 g-HIO, and day 28 g-HIO (panel A). Heatmap of spheroid and g-HIO samples (panel B).

FIG. 10C depicts an embodiment of biological processes transcriptionally enriched during g-HIO in vitro development. Enriched biological processes at day 0, day 6, day 14, and day 28 of g-HIO in vitro development are listed.

### DETAILED DESCRIPTION

Intestinal failure (IF) is usually a result of intestinal loss due to surgical resection and/or congenital bowel defects resulting in altered intestinal absorption and digestion. A smaller subgroup suffers from motility problems resulting in functional loss of the bowel's ability to absorb fluids and nutrients. Chronic intestinal failure occurs when the body is unable to maintain energy and nutritional needs through absorption of food or nutrients via the intestinal tract and which therefore necessitates long-term parenteral nutrition (PN). Intestinal failure affects about 3-50 people per million with ~15,000 people affected in the U.S. Chronic intestinal failure has been granted rare disease status under number ORPHA:294422 (classification: disorder).

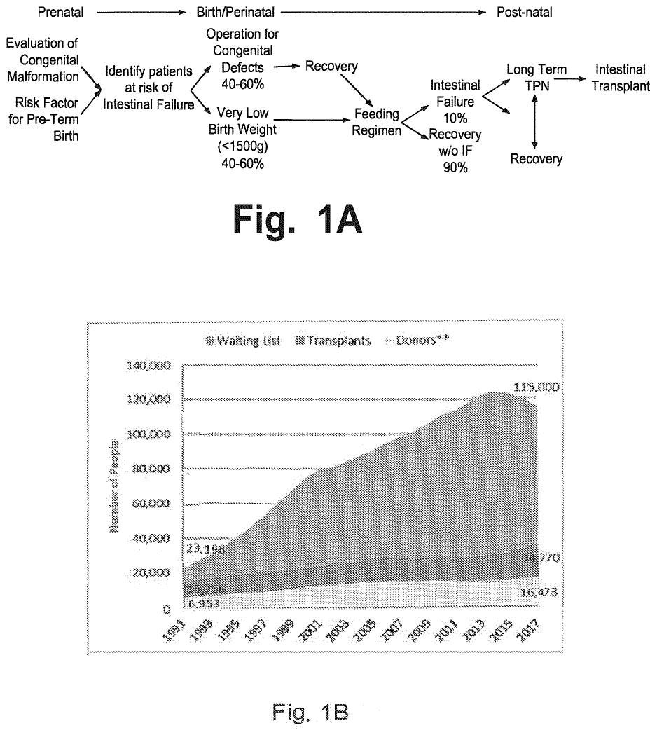

Long-term PN, while lifesaving, may lead to its own series of severe complications. In the neonatal population, PN-dependent intestinal failure can be associated with multiple complications including recurrent blood stream infections, repeat hospital admissions and subsequent poor growth and development including metabolic bone problems. All of these contribute to a very high burden on the patient, family, and health care system. Life-threatening complications in long-term PN patients such as inaccessible veins due to thrombosis, repeat catheter-related sepsis and cholestatic liver disease may eventually result in the need for life-rescuing intestinal transplantation. FIG. 1A shows the clinical progression from intestinal failure to intestinal transplantation.

Intestinal transplantation has evolved into an established therapeutic modality in the management of patients with irreversible intestinal failure. Intestinal transplantation can be performed in different forms, such as isolated intestinal transplant, modified multivisceral transplant and full multivisceral transplant. Even though the number of patients undergoing intestinal transplantation is much lower than other organ transplants, the number of procedures has increased 5-fold from 2000 to 2009. In the past few years, the number of intestinal transplants has stabilized and are trending down to rates between 100 and 120 cases per year with 47 pediatric transplants occurring in 2017. This trend is due to improved multidisciplinary care provided to intestinal failure patients, which will likely increase in population over the next decade. New and improved treatments are needed for this patient population.

While the surgical techniques continue to improve, the destructive alloimmunity of the intestinal and multivisceral transplant continues to be a significant obstacle that limits both the listing of potential patients that may benefit from a transplant and maintaining grafts after transplant. The acceptance of the Pittsburgh Protocol, which results in an initial complete depletion of T-cells immediately prior to transplant followed by a continued suppression of T-cells and placement on steroid maintenance, is considered standard of care. This protocol has decreased the rate of graft rejection, but this rate is still significant. Donor-specific antibodies (DSA) continue to be an issue in ~30% of patients in the early post-transplant period. The presence of DSAs more than double the risk of chronic rejection.

With one- and five-year survival rates above 70%, 10- and 15-year survival rates are only 42% and 35%, respectively. This indicates that while short-term outcomes are good, long-term results continue to be disappointing. In 2017, 90.4% of intestine only transplants were initial transplants while 9.6% were re-transplants. For those that received combined liver and intestine grafts, 26.3% of these were re-transplants. Intestinal re-transplantation is now the 4^{th} most common indication for intestinal transplant.

Pediatric patients undergoing re-transplantation tend to be younger than the primary transplantation cohort as a whole. One center's experience showed that the average time between primary and re-transplantation was 421 days. They found a bias towards survival in all patients who had early (<90 days) re-transplantation over those with late re-transplantation, 80% to 50% survival respectively. In the pediatric subpopulation, the re-transplantation 3-year survival rate was 27% regardless of timing, with the same percentage in graft survival. At a second center, out of 23 patients (both adult and pediatric) that underwent re-transplant, 15 patients died (35% survival) at a median period of 12 months after re-transplantation. A third center in Spain reported a 5-year re-transplantation pediatric survival rate of 35% in 13 patients.

When closely characterizing one re-transplantation cohort (n=23), recurrent severe rejection was common even after re-transplantation (35%). The incidence of graft rejection was significantly higher in the re-transplant if the patient had rejection with the primary transplant leading to graft loss and a 33% mortality rate. Re-transplanted patients were commonly severely immunocompromised with bone marrow suppression observed in 35% of re-transplantation patients compared to 4% of primary transplants. Additionally, those re-transplantation non-survivors (60%) had a significantly lower absolute lymphocyte and platelet counts one month prior to death compared to similar timepoints in survivors.

Organ donations continues to experience a significant shortage compared to need. As of July 2019, in the U.S., there are more than 113,000 men, women, and children on the national transplant waiting list (FIG. 1B). Every 10 minutes, a new person is added to the waiting list while 20 people die waiting on a transplant. This shortage has escalated over the last 27 years from 6,953 donors/23,198 waiting in 1991 to 17,554 donors/113,759 waiting in 2018.

This increasing scarcity of human organ donors has driven research scientists to examine other options such as xenotransplantation or to generate essential human transplantable organs. This approach not only has complicated scientific challenges, but also has legal and ethical issues. One potential option is the use of in vitro expanded epithelial biopsies obtained from the patient's own bowel (termed enteroids). As these structures contain only the epithelium, they cannot replace the majority of the bowel structure, which largely contains mesenchymal and neuronal tissues. In addition, these structures do not readily transplant, likely due to the lack of mesenchyme.

In contrast, organoids derived from induced pluripotent stem cells as described herein can contain both endodermal and mesodermal tissues, are readily transplantable, and contributed to regeneration of the entire graft. The ability to generate patient specific organoids may avoid several scientific and ethical concerns along with prevention of allogeneic immune response that could ultimately be the solution to helping individuals extend the life expectancy of their graft and avoid being placed back on the transplant waiting list.

Currently, organoids are being utilized to study human disease both in vitro and in vivo. As disclosed herein, there is strong data to support that these iPSC-derived tissues have the potential to fully function in vivo and therefore may serve to salvage organ transplants. It has been shown that pluripotent stem cells (PSCs) can be directed to differentiate into multiple organ systems in vitro including intestinal tissue by modulating the combinatorial activities of several signaling pathways in a stepwise fashion, effectively recapitulating the in vivo fetal organ development without the need of fetal tissue.

Disclosed herein are iPSC-derived gastrointestinal organoids. In some embodiments, the gastrointestinal organoid is an esophageal organoid, gastric organoid, intestinal organoid, or colon organoid. In some embodiments, the gastrointestinal organoid is an intestinal organoid. These organoids are generated from Sendai virus-transduced somatic cells induced into PSCs that can form all tissues of the body. By manipulating factors that control embryonic organogenesis, in vitro methods have been developed to guide the stepwise differentiation of PSC into embryonic germ layer restricted organoid, then specific cell types such as hepatocytes, neural, myocytes, and intestinal tissue. Methods of producing organoids such as intestinal organoids can be found in U.S. Pat. Nos. 9,719,068 and 10,174,289, and PCT Publications WO 2016/061464 and WO 2018/106628, each of which are hereby expressly incorporated by reference in its entirety.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

Unless defined otherwise, technical and scientific terms used herein have the same meaning as commonly understood when read in light of the instant disclosure by one of ordinary skill in the art to which the present disclosure belongs. For purposes of the present disclosure, the following terms are explained below.

The articles “a” and “an” are used herein to refer to one or to more than one (for example, at least one) of the grammatical object of the article. By way of example, “an element” means one element or more than one element.

By “about” is meant a quantity, level, value, number, frequency, percentage, dimension, size, amount, weight or length that varies by as much as 10% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

The terms “individual”, “subject”, or “patient” as used herein have their plain and ordinary meaning as understood in light of the specification, and mean a human or a non-human mammal, e.g., a dog, a cat, a mouse, a rat, a cow, a sheep, a pig, a goat, a non-human primate, or a bird, e.g., a chicken, as well as any other vertebrate or invertebrate. The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rodents, rats, mice, guinea pigs, or the like.

The terms “effective amount” or “effective dose” as used herein have their plain and ordinary meaning as understood in light of the specification, and refer to that amount of a recited composition or compound that results in an observable effect. Actual dosage levels of active ingredients in an active composition of the presently disclosed subject matter can be varied so as to administer an amount of the active composition or compound that is effective to achieve the desired response for a particular subject and/or application. The selected dosage level will depend upon a variety of factors including, but not limited to, the activity of the composition, formulation, route of administration, combination with other drugs or treatments, severity of the condition being treated, and the physical condition and prior medical history of the subject being treated. In some embodiments, a minimal dose is administered, and dose is escalated in the absence of dose-limiting toxicity to a minimally effective amount. Determination and adjustment of an effective dose, as well as evaluation of when and how to make such adjustments, are contemplated herein.

For clarity of disclosure, to the extent that spatial terms such as “upper”, “lower”, “longitudinal”, “lateral”, “transverse”, “inward”, “outward”, or the like are used herein or in reference to the drawings, it will be appreciated that such terms are used for exemplary description purposes only and are not intended to be limiting or absolute. In that regard, it will be understood that instruments such as those disclosed herein may be used in a variety of orientations and positions not limited to those shown and described herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm”.

The terms “function” and “functional” as used herein have their plain and ordinary meaning as understood in light of the specification, and refer to a biological, enzymatic, or therapeutic function.

The term “inhibit” as used herein has its plain and ordinary meaning as understood in light of the specification, and may refer to the reduction or prevention of a biological activity. The reduction can be by a percentage that is, is about, is at least, is at least about, is not more than, or is not more than about, 10%, 20%, 30%, 40%, 50%, 60%, 70%, 80%, 90%, or 100%, or an amount that is within a range defined by any two of the aforementioned values. As used herein, the term “delay” has its plain and ordinary meaning as understood in light of the specification, and refers to a slowing, postponement, or deferment of a biological event, to a time which is later than would otherwise be expected. The delay can be a delay of a percentage that is, is about, is at least, is at least about, is not more than, or is not more than about, 0%, 10%, 20%, 30%, 40%, 50%, 60%, 70%, 80%, 90%, 100%, or an amount within a range defined by any two of the aforementioned values. The terms inhibit and delay may not necessarily indicate a 100% inhibition or delay. A partial inhibition or delay may be realized.

As used herein, the term “isolated” has its plain and ordinary meaning as understood in light of the specification, and refers to a substance and/or entity that has been (1) separated from at least some of the components with which it was associated when initially produced (whether in nature and/or in an experimental setting), and/or (2) produced, prepared, and/or manufactured by the hand of man. Isolated substances and/or entities may be separated from equal to, about, at least, at least about, not more than, or not more than about, 10%, about 20%, about 30%, about 40%, about 50%, about 60%, about 70%, about 80%, about 90%, about 95%, about 98%, about 99%, substantially 100%, or 100% of the other components with which they were initially associated (or ranges including and/or spanning the aforementioned values). In some embodiments, isolated agents are, are about, are at least, are at least about, are not more than, or are not more than about 80%, about 85%, about 90%, about 91%, about 92%, about 93%, about 94%, about 95%, about 96%, about 97%, about 98%, about 99%, substantially 100%, or 100% pure (or ranges including and/or spanning the aforementioned values). As used herein, a substance that is “isolated” may be “pure” (e.g., substantially free of other components). As used herein, the term “isolated cell” may refer to a cell not contained in a multi-cellular organism or tissue.

As used herein, “in vivo” is given its plain and ordinary meaning as understood in light of the specification and refers to the performance of a method inside living organisms, usually animals, mammals, including humans, and plants, as opposed to a tissue extract or dead organism.

As used herein, “ex vivo” is given its plain and ordinary meaning as understood in light of the specification and refers to the performance of a method outside a living organism with little alteration of natural conditions.

As used herein, “in vitro” is given its plain and ordinary meaning as understood in light of the specification and refers to the performance of a method outside of biological conditions, e.g., in a petri dish or test tube.

The terms “nucleic acid” or “nucleic acid molecule” as used herein have their plain and ordinary meaning as understood in light of the specification, and refer to polynucleotides, such as deoxyribonucleic acid (DNA) or ribonucleic acid (RNA), oligonucleotides, those that appear in a cell naturally, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, or phosphoramidate. The term “nucleic acid molecule” also includes so-called “peptide nucleic acids,” which comprise naturally-occurring or modified nucleic acid bases attached to a polyamide backbone. Nucleic acids can be either single stranded or double stranded. “Oligonucleotide” can be used interchangeable with nucleic acid and can refer to either double stranded or single stranded DNA or RNA. A nucleic acid or nucleic acids can be contained in a nucleic acid vector or nucleic acid construct (e.g. plasmid, virus, retrovirus, lentivirus, bacteriophage, cosmid, fosmid, phagemid, bacterial artificial chromosome (BAC), yeast artificial chromosome (YAC), or human artificial chromosome (HAC)) that can be used for amplification and/or expression of the nucleic acid or nucleic acids in various biological systems. Typically, the vector or construct will also contain elements including but not limited to promoters, enhancers, terminators, inducers, ribosome binding sites, translation initiation sites, start codons, stop codons, polyadenylation signals, origins of replication, cloning sites, multiple cloning sites, restriction enzyme sites, epitopes, reporter genes, selection markers, antibiotic selection markers, targeting sequences, peptide purification tags, or accessory genes, or any combination thereof.

A nucleic acid or nucleic acid molecule can comprise one or more sequences encoding different peptides, polypeptides, or proteins. These one or more sequences can be joined in the same nucleic acid or nucleic acid molecule adjacently, or with extra nucleic acids in between, e.g. linkers, repeats or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a nucleic acid as used herein has its plain and ordinary meaning as understood in light of the specification and refers to a sequence being after the 3′-end of a previous sequence, on the strand containing the encoding sequence (sense strand) if the nucleic acid is double stranded. The term “upstream” on a nucleic acid as used herein has its plain and ordinary meaning as understood in light of the specification and refers to a sequence being before the 5′-end of a subsequent sequence, on the strand containing the encoding sequence (sense strand) if the nucleic acid is double stranded. The term “grouped” on a nucleic acid as used herein has its plain and ordinary meaning as understood in light of the specification and refers to two or more sequences that occur in proximity either directly or with extra nucleic acids in between, e.g. linkers, repeats, or restriction enzyme sites, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths, but generally not with a sequence in between that encodes for a functioning or catalytic polypeptide, protein, or protein domain.

The nucleic acids described herein comprise nucleobases. Primary, canonical, natural, or unmodified bases are adenine, cytosine, guanine, thymine, and uracil. Other nucleobases include but are not limited to purines, pyrimidines, modified nucleobases, 5-methylcytosine, pseudouridine, dihydrouridine, inosine, 7-methylguanosine, hypoxanthine, xanthine, 5,6-dihydrouracil, 5-hydroxymethylcytosine, 5-bromouracil, isoguanine, isocytosine, aminoallyl bases, dye-labeled bases, fluorescent bases, or biotin-labeled bases.

The terms “peptide”, “polypeptide”, and “protein” as used herein have their plain and ordinary meaning as understood in light of the specification and refer to macromolecules comprised of amino acids linked by peptide bonds. The numerous functions of peptides, polypeptides, and proteins are known in the art, and include but are not limited to enzymes, structure, transport, defense, hormones, or signaling. Peptides, polypeptides, and proteins are often, but not always, produced biologically by a ribosomal complex using a nucleic acid template, although chemical syntheses are also available. By manipulating the nucleic acid template, peptide, polypeptide, and protein mutations such as substitutions, deletions, truncations, additions, duplications, or fusions of more than one peptide, polypeptide, or protein can be performed. These fusions of more than one peptide, polypeptide, or protein can be joined in the same molecule adjacently, or with extra amino acids in between, e.g. linkers, repeats, epitopes, or tags, or any other sequence that is, is about, is at least, is at least about, is not more than, or is not more than about, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, or 300 bases long, or any length in a range defined by any two of the aforementioned lengths. The term “downstream” on a polypeptide as used herein has its plain and ordinary meaning as understood in light of the specification and refers to a sequence being after the C-terminus of a previous sequence. The term “upstream” on a polypeptide as used herein has its plain and ordinary meaning as understood in light of the specification and refers to a sequence being before the N-terminus of a subsequent sequence.

The term “purity” of any given substance, compound, or material as used herein has its plain and ordinary meaning as understood in light of the specification and refers to the actual abundance of the substance, compound, or material relative to the expected abundance. For example, the substance, compound, or material may be at least 80, 85, 90, 91, 92, 93, 94, 95, 96, 97, 98, 99, or 100% pure, including all decimals in between. Purity may be affected by unwanted impurities, including but not limited to nucleic acids, DNA, RNA, nucleotides, proteins, polypeptides, peptides, amino acids, lipids, cell membrane, cell debris, small molecules, degradation products, solvent, carrier, vehicle, or contaminants, or any combination thereof. In some embodiments, the substance, compound, or material is substantially free of host cell proteins, host cell nucleic acids, plasmid DNA, contaminating viruses, proteasomes, host cell culture components, process related components, mycoplasma, pyrogens, bacterial endotoxins, and adventitious agents. Purity can be measured using technologies including but not limited to electrophoresis, SDS-PAGE, capillary electrophoresis, PCR, rtPCR, qPCR, chromatography, liquid chromatography, gas chromatography, thin layer chromatography, enzyme-linked immunosorbent assay (ELISA), spectroscopy, UV-visible spectrometry, infrared spectrometry, mass spectrometry, nuclear magnetic resonance, gravimetry, or titration, or any combination thereof.

The term “yield” of any given substance, compound, or material as used herein has its plain and ordinary meaning as understood in light of the specification and refers to the actual overall amount of the substance, compound, or material relative to the expected overall amount. For example, the yield of the substance, compound, or material is, is about, is at least, is at least about, is not more than, or is not more than about, 80, 85, 90, 91, 92, 93, 94, 95, 96, 97, 98, 99, or 100% of the expected overall amount, including all decimals in between. Yield may be affected by the efficiency of a reaction or process, unwanted side reactions, degradation, quality of the input substances, compounds, or materials, or loss of the desired substance, compound, or material during any step of the production.

The term “% w/w” or “% wt/wt” as used herein has its plain and ordinary meaning as understood in light of the specification and refers to a percentage expressed in terms of the weight of the ingredient or agent over the total weight of the composition multiplied by 100. The term “% v/v” or “% vol/vol” as used herein has its plain and ordinary meaning as understood in the light of the specification and refers to a percentage expressed in terms of the liquid volume of the compound, substance, ingredient, or agent over the total liquid volume of the composition multiplied by 100.

### Stem Cells

The term “totipotent stem cells” (also known as omnipotent stem cells) as used herein has its plain and ordinary meaning as understood in light of the specification and are stem cells that can differentiate into embryonic and extra-embryonic cell types. Such cells can construct a complete, viable organism. These cells are produced from the fusion of an egg and sperm cell. Cells produced by the first few divisions of the fertilized egg are also totipotent.

The term “embryonic stem cells (ESCs),” also commonly abbreviated as ES cells, as used herein has its plain and ordinary meaning as understood in light of the specification and refers to cells that are pluripotent and derived from the inner cell mass of the blastocyst, an early-stage embryo. For purpose of the present invention, the term “ESCs” is used broadly sometimes to encompass the embryonic germ cells as well.

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes. Pluripotent stem cells can be derived from any suitable source. Examples of sources of pluripotent stem cells include mammalian sources, including human, rodent, porcine, and bovine.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., Sox1, Sox2, Sox3, and Sox15); certain members of the Klf family (e.g., Klf1, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, β-Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

The term “precursor cell” as used herein has its plain and ordinary meaning as understood in light of the specification and encompasses any cells that can be used in methods described herein, through which one or more precursor cells acquire the ability to renew itself or differentiate into one or more specialized cell types. In some embodiments, a precursor cell is pluripotent or has the capacity to becoming pluripotent. In some embodiments, the precursor cells are subjected to the treatment of external factors (e.g., growth factors) to acquire pluripotency. In some embodiments, a precursor cell can be a totipotent (or omnipotent) stem cell; a pluripotent stem cell (induced or non-induced); a multipotent stem cell; an oligopotent stem cells and a unipotent stem cell. In some embodiments, a precursor cell can be from an embryo, an infant, a child, or an adult. In some embodiments, a precursor cell can be a somatic cell subject to treatment such that pluripotency is conferred via genetic manipulation or protein/peptide treatment. Precursor cells include embryonic stem cells (ESC), embryonic carcinoma cells (ECs), and epiblast stem cells (EpiSC).

In some embodiments, one step is to obtain stem cells that are pluripotent or can be induced to become pluripotent. In some embodiments, pluripotent stem cells are derived from embryonic stem cells, which are in turn derived from totipotent cells of the early mammalian embryo and are capable of unlimited, undifferentiated proliferation in vitro. Embryonic stem cells are pluripotent stem cells derived from the inner cell mass of the blastocyst, an early-stage embryo. Methods for deriving embryonic stem cells from blastocytes are well known in the art. Human embryonic stem cells H9 (H9-hESCs) are used in the exemplary embodiments described in the present application, but it would be understood by one of skill in the art that the methods and systems described herein are applicable to any stem cells.

Additional stem cells that can be used in embodiments in accordance with the present invention include but are not limited to those provided by or described in the database hosted by the National Stem Cell Bank (NSCB), Human Embryonic Stem Cell Research Center at the University of California, San Francisco (UCSF); WISC cell Bank at the Wi Cell Research Institute; the University of Wisconsin Stem Cell and Regenerative Medicine Center (UW-SCRMC); Novocell, Inc. (San Diego, Calif.); Cellartis AB (Goteborg, Sweden); ES Cell International Pte Ltd (Singapore); Technion at the Israel Institute of Technology (Haifa, Israel); and the Stem Cell Database hosted by Princeton University and the University of Pennsylvania. Exemplary embryonic stem cells that can be used in embodiments in accordance with the present invention include but are not limited to SA01 (SA001); SA02 (SA002); ES01 (HES-1); ES02 (HES-2); ES03 (HES-3); ES04 (HES-4); ES05 (HES-5); ES06 (HES-6); BG01 (BGN-01); BG02 (BGN-02); BG03 (BGN-03); TE03 (13); TE04 (14); TE06 (16); UCO1 (HSF1); UC06 (HSF6); WA01 (HI); WA07 (H7); WA09 (H9); WA13 (H13); WA14 (H14). Exemplary human pluripotent cell lines include but are not limited to TkDA3-4, 1231A3, 317-D6, 317-A4, CDH1, 5-T-3, 3-34-1, NAFLD27, NAFLD77, NAFLD150, WD90, WD91, WD92, L20012, C213, 1383D6, FF, or 317-12 cells.

In developmental biology, cellular differentiation is the process by which a less specialized cell becomes a more specialized cell type. As used herein, the term “directed differentiation” describes a process through which a less specialized cell becomes a particular specialized target cell type. The particularity of the specialized target cell type can be determined by any applicable methods that can be used to define or alter the destiny of the initial cell. Exemplary methods include but are not limited to genetic manipulation, chemical treatment, protein treatment, and nucleic acid treatment.

In some embodiments, an adenovirus can be used to transport the requisite four genes, resulting in iPSCs substantially identical to embryonic stem cells. Since the adenovirus does not combine any of its own genes with the targeted host, the danger of creating tumors is eliminated. In some embodiments, non-viral based technologies are employed to generate iPSCs. In some embodiments, reprogramming can be accomplished via plasmid without any virus transfection system at all, although at very low efficiencies. In other embodiments, direct delivery of proteins is used to generate iPSCs, thus eliminating the need for viruses or genetic modification. In some embodiment, generation of mouse iPSCs is possible using a similar methodology: a repeated treatment of the cells with certain proteins channeled into the cells via poly-arginine anchors was sufficient to induce pluripotency. In some embodiments, the expression of pluripotency induction genes can also be increased by treating somatic cells with FGF2 under low oxygen conditions.

The term “Sendai virus” as used herein has its plain and ordinary meaning as understood in light of the specification and refers to an enveloped, negative-sense, single-stranded RNA virus of the family Paramyxoviridae and is also known as murine parainfluenza virus type 1 or hemagglutinating virus of Japan (HVJ). While typically only disease-causing in rodents, the virus can infect a wide range of mammalian cells, including human cells, by the sialic acid receptor. Sendai virus can be used as a viral vector to deliver transgenes to cells in vitro and in vivo. In some embodiments, to reprogram somatic cells to induced pluripotent stem cells, Sendai virus have been engineered to comprise expression vectors for stem cell reprogramming factors. In some embodiments, the stem cell reprogramming factors include but are not limited to Oct3/4, Sox2, Klf4, and L-Myc, or any combination thereof, but can also include any stem cell reprogramming factor disclosed herein or known in the art. In some embodiments, these stem cell reprogramming factors are human in origin. In some embodiments, a Sendai virus vector comprises expression vectors for one or more (e.g. at least 1, 2, 3, 4, 5) of Oct3/4, Sox2, Klf4, L-Myc, or another stem cell reprogramming factor. In some embodiments, a Sendai virus vector comprises an expression vector for Klf4, Oct3/4 and Sox2 (KOS). In some embodiments, a Sendai virus vector comprises an expression vector for L-Myc. In some embodiments, a Sendai virus vector comprises an expression vector for Klf4. In some embodiments, one or more Sendai virus vectors are combined in different ratios to optimize reprogramming of cells. In some embodiments, contacting a somatic cell with one or more Sendai virus vectors successfully reprograms the somatic cell to an induced pluripotent stem cell. As an RNA virus, Sendai virus does not require integration of the viral payload into the host genome nor does it require access to the nucleus (like DNA viruses). This differs from lentiviruses and adenoviruses. However, it is envisioned that other viral vectors such as lentiviruses, adenoviruses, and adeno-associated viruses can be used for transduction purposes described herein where Sendai viruses are used, such as for reprogramming somatic cells to stem cells.

The term “feeder cell” as used herein has its plain and ordinary meaning as understood in light of the specification and refers to cells that support the growth of pluripotent stem cells, such as by secreting growth factors into the medium or displaying on the cell surface. Feeder cells are generally adherent cells and may be growth arrested. For example, feeder cells are growth-arrested by irradiation (e.g. gamma rays), mitomycin-C treatment, electric pulses, or mild chemical fixation (e.g. with formaldehyde or glutaraldehyde). However, feeder cells do not necessarily have to be growth arrested. Feeder cells may serve purposes such as secreting growth factors, displaying growth factors on the cell surface, detoxifying the culture medium, or synthesizing extracellular matrix proteins. In some embodiments, the feeder cells are allogeneic or xenogeneic to the supported target stem cell, which may have implications in downstream applications. In some embodiments, the feeder cells are mouse cells. In some embodiments, the feeder cells are human cells. In some embodiments, the feeder cells are mouse fibroblasts, mouse embryonic fibroblasts, mouse STO cells, mouse 3T3 cells, mouse SNL 76/7 cells, human fibroblasts, human foreskin fibroblasts, human dermal fibroblasts, human adipose mesenchymal cells, human bone marrow mesenchymal cells, human amniotic mesenchymal cells, human amniotic epithelial cells, human umbilical cord mesenchymal cells, human fetal muscle cells, human fetal fibroblasts, or human adult fallopian tube epithelial cells. In some embodiments, conditioned medium prepared from feeder cells is used in lieu of feeder cell co-culture or in combination with feeder cell co-culture. In some embodiments, feeder cells are not used during the proliferation of the target stem cells.

Some embodiments described herein relate to pharmaceutical compositions that comprise, consist essentially of, or consist of an effective amount of a cell composition described herein and a pharmaceutically acceptable carrier, excipient, or combination thereof. A pharmaceutical composition described herein is suitable for human and/or veterinary applications.

As used herein, “pharmaceutically acceptable” has its plain and ordinary meaning as understood in light of the specification and refers to carriers, excipients, and/or stabilizers that are nontoxic to the cell or mammal being exposed thereto at the dosages and concentrations employed or that have an acceptable level of toxicity. A “pharmaceutically acceptable” “diluent,” “excipient,” and/or “carrier” as used herein have their plain and ordinary meaning as understood in light of the specification and are intended to include any and all solvents, dispersion media, coatings, antibacterial and antifungal agents, isotonic and absorption delaying agents, and the like, compatible with administration to humans, cats, dogs, or other vertebrate hosts. Typically, a pharmaceutically acceptable diluent, excipient, and/or carrier is a diluent, excipient, and/or carrier approved by a regulatory agency of a Federal, a state government, or other regulatory agency, or listed in the U.S. Pharmacopeia or other generally recognized pharmacopeia for use in animals, including humans as well as non-human mammals, such as cats and dogs. The term diluent, excipient, and/or “carrier” can refer to a diluent, adjuvant, excipient, or vehicle with which the pharmaceutical composition is administered. Such pharmaceutical diluent, excipient, and/or carriers can be sterile liquids, such as water and oils, including those of petroleum, animal, vegetable or synthetic origin. Water, saline solutions and aqueous dextrose and glycerol solutions can be employed as liquid diluents, excipients, and/or carriers, particularly for injectable solutions. Suitable pharmaceutical diluents and/or excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A non-limiting example of a physiologically acceptable carrier is an aqueous pH buffered solution. The physiologically acceptable carrier may also comprise one or more of the following: antioxidants, such as ascorbic acid, low molecular weight (less than about 10 residues) polypeptides, proteins, such as serum albumin, gelatin, immunoglobulins, hydrophilic polymers such as polyvinylpyrrolidone, amino acids, carbohydrates such as glucose, mannose, or dextrins, chelating agents such as EDTA, sugar alcohols such as mannitol or sorbitol, salt-forming counterions such as sodium, and nonionic surfactants such as TWEEN®, polyethylene glycol (PEG), and PLURONICS®. The composition, if desired, can also contain minor amounts of wetting, bulking, emulsifying agents, or pH buffering agents. These compositions can take the form of solutions, suspensions, emulsion, sustained release formulations and the like. The formulation should suit the mode of administration.

Cryoprotectants are cell composition additives to improve efficiency and yield of low temperature cryopreservation by preventing formation of large ice crystals. Cryoprotectants include but are not limited to DMSO, ethylene glycol, glycerol, propylene glycol, trehalose, formamide, methyl-formamide, dimethyl-formamide, glycerol 3-phosphate, proline, sorbitol, diethyl glycol, sucrose, triethylene glycol, polyvinyl alcohol, polyethylene glycol, or hydroxyethyl starch. Cryoprotectants can be used as part of a cryopreservation medium, which include other components such as nutrients (e.g. albumin, serum, bovine serum, fetal calf serum [FCS]) to enhance post-thawing survivability of the cells. In these cryopreservation media, at least one cryoprotectant may be found at a concentration that is, is about, is at least, is at least about, is not more than, or is not more than about, 0.01%, 0.05%, 0.1%, 0.5%, 1%, 2%, 3%, 4%, 5%, 6%, 7%, 8%, 9%, 10%, 11%, 12%, 13%, 14%, 15%, 16%, 17%, 18%, 19%, 20%, 30%, 40%, 50%, 60%, 70%, 80%, or 90%, or any percentage within a range defined by any two of the aforementioned numbers.

Additional excipients with desirable properties include but are not limited to preservatives, adjuvants, stabilizers, solvents, buffers, diluents, solubilizing agents, detergents, surfactants, chelating agents, antioxidants, alcohols, ketones, aldehydes, ethylenediaminetetraacetic acid (EDTA), citric acid, salts, sodium chloride, sodium bicarbonate, sodium phosphate, sodium borate, sodium citrate, potassium chloride, potassium phosphate, magnesium sulfate sugars, dextrose, fructose, mannose, lactose, galactose, sucrose, sorbitol, cellulose, serum, amino acids, polysorbate 20, polysorbate 80, sodium deoxycholate, sodium taurodeoxycholate, magnesium stearate, octylphenol ethoxylate, benzethonium chloride, thimerosal, gelatin, esters, ethers, 2-phenoxyethanol, urea, or vitamins, or any combination thereof. Some excipients may be in residual amounts or contaminants from the process of manufacturing, including but not limited to serum, albumin, ovalbumin, antibiotics, inactivating agents, formaldehyde, glutaraldehyde, 0-propiolactone, gelatin, cell debris, nucleic acids, peptides, amino acids, or growth medium components or any combination thereof. The amount of the excipient may be found in composition at a percentage that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 20%, 30%, 40%, 50%, 60%, 70%, 80%, 90%, 95%, 100% w/w or any percentage by weight in a range defined by any two of the aforementioned numbers.

The term “pharmaceutically acceptable salts” has its plain and ordinary meaning as understood in light of the specification and includes relatively non-toxic, inorganic and organic acid, or base addition salts of compositions or excipients, including without limitation, analgesic agents, therapeutic agents, other materials, and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc, and the like. Salts may also be formed with suitable organic bases, including those that are non-toxic and strong enough to form such salts. For example, the class of such organic bases may include but are not limited to mono-, di-, and trialkylamines, including methylamine, dimethylamine, and triethylamine; mono-, di-, or trihydroxyalkylamines including mono-, di-, and triethanolamine; amino acids, including glycine, arginine and lysine; guanidine; N-methylglucosamine; N-methylglucamine; L-glutamine; N-methylpiperazine; morpholine; ethylenediamine; N-benzylphenethylamine; trihydroxymethyl aminoethane.

Proper formulation is dependent upon the route of administration chosen. Techniques for formulation and administration of the compounds described herein are known to those skilled in the art. Multiple techniques of administering a compound exist in the art including, but not limited to, enteral, oral, rectal, topical, sublingual, buccal, intraaural, epidural, epicutaneous, aerosol, parenteral delivery, including intramuscular, subcutaneous, intra-arterial, intravenous, intraportal, intra-articular, intradermal, peritoneal, intramedullary injections, intrathecal, direct intraventricular, intraperitoneal, intranasal or intraocular injections. Pharmaceutical compositions will generally be tailored to the specific intended route of administration.

As used herein, a “carrier” has its plain and ordinary meaning as understood in light of the specification and refers to a compound, particle, solid, semi-solid, liquid, or diluent that facilitates the passage, delivery and/or incorporation of a compound to cells, tissues and/or bodily organs.

As used herein, a “diluent” has its plain and ordinary meaning as understood in light of the specification and refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

The invention is generally disclosed herein using affirmative language to describe the numerous embodiments. The invention also includes embodiments in which subject matter is excluded, in full or in part, such as substances or materials, method steps and conditions, protocols, or procedures.

### Intestinal Organ Development

In anatomy, the intestine (or bowel) is the segment of the alimentary canal extending from the stomach to the anus and, in humans and other mammals, consists of two segments, the small intestine and the large intestine. In humans, the small intestine is further subdivided into the duodenum, jejunum and ileum while the large intestine is subdivided into the cecum and colon. The structure of an intestinal organ is described herein using the human organ as an example. It will be understood by one of ordinary skill in the art that the methods and systems described herein are applicable to the intestinal systems of all mammals.

The intestinal tract can be broadly divided into two different parts, the small and large intestine. Grayish-purple in color and about 35 millimeters (1.5 inches) in diameter, the small intestine is the first and longer, measuring 6 to 7 meters (20-23 feet) long average in an adult man. Shorter and relatively stockier, the large intestine is a dark reddish color, measuring roughly 1.5 meters (5 feet) long on average.

The lumen is the cavity where digested food passes through and from where nutrients are absorbed. Both intestines share a general structure with the whole gut, and are composed of several layers.

Going from inside the lumen radially outwards, the order proceeds from the mucosa (epithelium and muscularis mucosa), submucosa, muscularis externa (made up of inner circular and outer longitudinal), and lastly serosa. Along the whole length of the gut in the epithelium are goblet cells. These secrete mucus which lubricates the passage of food and protects the gut from digestive enzymes. Crypts are invaginations of the mucosa and villi are finger-like projections that increase the overall surface area of the intestine while also containing a lacteal, which is connected to the lymph system and aids in the removal of lipids and tissue fluid from the blood supply. During development, the epithelium buckles and invaginations occur resulting in ridges that later resolve into a crypt-villus architecture. Microvilli are present on the epithelium of a villus and further increase the surface area over which absorption can take place. The muscularis mucosa is a layer of smooth muscle that aids in the action of continued peristalsis and catastalsis along the gut. The submucosa contains nerves (e.g., Meissner's plexus), blood vessels and elastic fibers with collagen that stretches with increased capacity but maintains the shape of the intestine. The muscularis externa comprises longitudinal and smooth muscle that again helps with continued peristalsis and the movement of digested material out of and along the gut. In between the two layers of muscle lies Auerbach's plexus. The serosa is made up of loose connective tissue and coated in mucus so as to prevent friction damage from the intestine rubbing against other tissue. Holding all this in place are the mesenteries which suspend the intestine in the abdominal cavity and stop it from being disturbed when a person is physically active.

### Differentiation of PSCs

In some embodiments, PSCs, such as ESCs and iPSCs, undergo directed differentiation in a stepwise manner first into definitive endoderm (DE) then into posterior/hindgut epithelium (e.g., hindgut spheroids), and then into intestinal tissue. In some embodiments, PSCs, such as ESCs and iPSCs, undergo directed differentiation in a non-stepwise manner where molecules (e.g., growth factors, ligands) for promoting DE formation and those for subsequent tissue formation are added at the same time.

The definitive endoderm gives rise to the gut tube. The anterior DE forms the foregut and its associated organs including the esophagus, lungs, stomach, liver and pancreas and the posterior DE forms the midgut and hindgut, which forms the small and large intestines and parts of the genitourinary system. Studies using mouse, chick and frog embryos suggest that establishing the anterior-posterior pattern in DE at the gastrula stage is a prerequisite for subsequent foregut and hindgut development. The Wnt and FGF signaling pathways are critical for promoting either posterior endoderm/hindgut or anterior endoderm/foregut fate. In hindgut, the simple cuboidal epithelium first develops into a pseudostratified columnar epithelium, then into villi containing a polarized columnar epithelium and a proliferative zone at the base of the villi, which corresponds with the presumptive progenitor domain.

A robust and efficient process to direct the differentiation of DE into intestinal tissue in vitro has been previously described in U.S. Pat. No. 9,719,068. In some embodiments, directed differentiation is achieved by selectively activating certain signaling pathways in the iPSCs and/or DE cells. In some embodiments, the signaling pathways are those active in intestinal development, including but not limited to the Wnt signaling pathway; Wnt/APC signaling pathway; FGF signaling pathway; TGF-beta signaling pathway; BMP signaling pathway; Notch signaling pathway; Hedgehog signaling pathway; LKB signaling pathway; and Par polarity signaling pathway.

Any methods for producing definitive endoderm from pluripotent cells (e.g., iPSCs or ESCs) are applicable to the methods described herein. In some embodiments, pluripotent cells are derived from a morula. In some embodiments, pluripotent stem cells are stem cells. Stem cells used in these methods can include, but are not limited to, embryonic stem cells. Embryonic stem cells can be derived from the embryonic inner cell mass or from the embryonic gonadal ridges. Embryonic stem cells or germ cells can originate from a variety of animal species including, but not limited to, various mammalian species including humans. In some embodiments, human embryonic stem cells are used to produce definitive endoderm. In some embodiments, human embryonic germ cells are used to produce definitive endoderm. In some embodiments, iPSCs are used to produce definitive endoderm. In some embodiments, human iPSCs (hiPSCs) are used to produce definitive endoderm.

In some embodiments, the embryonic stem cells or germ cells or iPSCs are treated with one or more small molecule compounds, activators, inhibitors, or growth factors for a time that is, is about, is at least, is at least about, is not more than, or is not more than about, 6 hours, 12 hours, 18 hours, 24 hours, 36 hours, 48 hours, 60 hours, 72 hours, 84 hours, 96 hours, 120 hours, 150 hours, 180 hours, 240 hours, 300 hours or any time within a range defined by any two of the aforementioned times, for example 6 hours to 300 hours, 24 hours to 120 hours, 48 hours to 96 hours, 6 hours to 72 hours, or 24 hours to 300 hours. In some embodiments, more than one small molecule compounds, activators, inhibitors, or growth factors are added. In these cases, the more than one small molecule compounds, activators, inhibitors, or growth factors can be added simultaneously or separately.

In some embodiments, the embryonic stem cells or germ cells or iPSCs are treated with one or more small molecule compounds, activators, inhibitors, or growth factors at a concentration that is, is about, is at least, is at least about, is not more than, or is not more than about, 10 ng/mL, 20 ng/mL, 50 ng/mL, 75 ng/mL, 100 ng/mL, 120 ng/mL, 150 ng/mL, 200 ng/mL, 500 ng/mL, 1000 ng/mL, 1200 ng/mL, 1500 ng/mL, 2000 ng/mL, 5000 ng/mL, 7000 ng/mL, 10000 ng/mL, or 15000 ng/mL, or any concentration that is within a range defined by any two of the aforementioned concentrations, for example, 10 ng/mL to 15000 ng/mL, 100 ng/mL to 5000 ng/mL, 500 ng/mL to 2000 ng/mL, 10 ng/mL to 2000 ng/mL, or 1000 ng/mL to 15000 ng/mL. In some embodiments, concentration of the one or more small molecule compounds, activators, inhibitors, or growth factors is maintained at a constant level throughout the treatment. In some embodiments, concentration of the one or more small molecule compounds, activators, inhibitors, or growth factors is varied during the course of the treatment. In some embodiments, more than one small molecule compounds, activators, inhibitors, or growth factors are added. In these cases, the more than one small molecule compounds, activators, inhibitors, or growth factors can differ in concentrations.

In some embodiments, the ESCs, germ cells, or iPSCs are cultured in growth media that supports the growth of stem cells. In some embodiments, the ESCs, germ cells, or iPSCs are cultured in stem cell growth media. In some embodiments, the stem cell growth media is RPMI 1640, DMEM, DMEM/F12, Erythroid Expansion Media, Minigut media, StemPro 34 SFM (serum free media), StemPro hESC SFM, mTeSR 1, or mTeSR Plus media. In some embodiments, the stem cell growth media comprises fetal bovine serum (FBS). In some embodiments, the stem cell growth media comprises FBS at a concentration that is, is about, is at least, is at least about, is not more than, or is not more than about, 0%, 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, 11%, 12%, 13%, 14%, 15%, 16%, 17%, 18%, 19%, or 20%, or any percentage within a range defined by any two of the aforementioned concentrations, for example 0% to 20%, 0.2% to 10%, 2% to 5%, 0% to 5%, or 2% to 20%. In some embodiments, the stem cell growth media does not contain xenogeneic components. In some embodiments, the growth media comprises one or more small molecule compounds, activators, inhibitors, or growth factors.

In some embodiments, populations of cells enriched in definitive endoderm cells are used. In some embodiments, the definitive endoderm cells are isolated or substantially purified. In some embodiments, the isolated or substantially purified definitive endoderm cells express one or more (e.g. at least 1, 3) of SOX17, FOXA2, or CXRC4 markers to a greater extent than one or more (e.g. at least 1, 3, 5) of OCT4, AFP, TM, SPARC, or SOX7 markers.

Methods for enriching a cell population with definitive endoderm are also contemplated. In some embodiments, definitive endoderm cells can be isolated or substantially purified from a mixed cell population by contacting the cells with a reagent that binds to a molecule that is present on the surface of definitive endoderm cells but which is not present on the surface of other cells in the mixed cell population, and then isolating the cells bound to the reagent. In certain embodiments, the cellular constituent that is present on the surface of definitive endoderm cells is CXCR4.

Some embodiments relate to CXCR4 antibodies, SDF-1 protein or ligands or other protein or ligands for CXCR4 can be used to obtain definitive endoderm cells in an enriched, isolated or substantially purified form. For example, a CXCR4 antibody, an SDF-1 protein or ligand or another protein or ligand for CXCR4 can be used as a reagent in a method, such as affinity-based separation or magnetic-based separation, to enrich, isolate or substantially purify preparations of definitive endoderm cells that bind to the reagent.

In some embodiments, definitive endoderm cells and hESCs are treated with one or more growth factors. Such growth factors can include growth factors from the TGF-beta superfamily. In some embodiments, the one or more growth factors comprise the Nodal/Activin and/or the BMP subgroups of the TGF-beta superfamily of growth factors. In some embodiments, the one or more growth factors are selected from the group consisting of Nodal, Activin A, Activin B, BMP4, Wnt3a or combinations of any of these growth factors.

In some embodiments, activin-induced definitive endoderm (DE) can further undergo FGF/Wnt induced posterior endoderm pattering, hindgut specification and morphogenesis, and finally a pro-intestinal culture system that promoted intestinal growth, morphogenesis and cytodifferentiation into functional intestinal cell types including mesenchyme, enterocytes, goblet, Paneth and enteroendocrine cells. In some embodiments, human PSCs are efficiently directed to differentiate in vitro into intestinal epithelium that includes secretory, endocrine and absorptive cell types. It will be understood that molecules such as growth factors can be added to any stage of the development to promote a particular type of intestinal tissue formation.

In some embodiments, FGF and Wnt proteins or ligands are used to mimic early hindgut specification in culture to convert, through directed differentiation, DE developed from iPSCs or ESCs into hindgut epithelium that efficiently gives rise to all the major intestinal cell types. In human, directed differentiation of DE is achieved through selective activating certain signaling pathways that are important to intestinal development.

Human intestinal development in vitro occurs in stages that approximate fetal gut development; endoderm formation, posterior endoderm patterning, hindgut morphogenesis, fetal gut development, epithelial morphogenesis, formation of a presumptive progenitor domain, and differentiation into functional cell types of the intestine. For example, in human, genes that encode Wnt signaling proteins include but are not limited to Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16.

It will be understood by one of skill in the art that altering the concentration, expression or function of one or more Wnt signaling proteins in combination with altering the concentration, expression, or function of one or more FGF proteins can give rise to directed differentiation in accordance of the present invention. In some embodiments, cellular constituents associated with the Wnt and/or FGF signaling pathways, for example, natural inhibitors, antagonists, activators, or agonists of the pathways can be used to result in inhibition or activation of the Wnt and/or FGF signaling pathways. In some embodiments, siRNA and/or shRNA targeting cellular constituents associated with the Wnt and/or FGF signaling pathways are used to inhibit or activate these pathways.

Fibroblast growth factors (FGFs) are a family of growth factors involved in angiogenesis, wound healing, and embryonic development. The FGFs are heparin-binding proteins and interactions with cell-surface associated heparan sulfate proteoglycans have been shown to be essential for FGF signal transduction. FGFs are key players in the processes of proliferation and differentiation of wide variety of cells and tissues. In humans, 22 members of the FGF family have been identified, all of which are structurally related signaling molecules. Members FGF1 through FGF10 all bind fibroblast growth factor receptors (FGFRs). FGF1 is also known as acidic, and FGF2 is also known as basic fibroblast growth factor (bFGF). Members FGF11, FGF12, FGF13, and FGF14, also known as FGF homologous factors 1-4 (FHF1-FHF4), have been shown to have distinct functional differences compared to the FGFs. Although these factors possess remarkably similar sequence homology, they do not bind FGFRs and are involved in intracellular processes unrelated to the FGFs. This group is also known as “iFGF.” Members FGF15 through FGF23 are newer and not as well characterized. FGF15 is the mouse ortholog of human FGF19 (hence there is no human FGF15). Human FGF20 was identified based on its homology to Xenopus FGF-20 (XFGF-20). In contrast to the local activity of the other FGFs, FGF15/FGF19, FGF21 and FGF23 have more systemic effects.

In some embodiments, it will be understood by one of skill in the art that any of the FGFs can be used in conjunction with a protein from the Wnt signaling pathway. In some embodiments, the FGF used is one or more of FGF1, FGF2, FGF3, FGF4, FGF4, FGF5, FGF6, FGF7, FGF8, FGF8, FGF9, FGF10, FGF11, FGF12, FGF13, FGF14, FGF15 (FGF19, FGF15/FGF19), FGF16, FGF17, FGF18, FGF20, FGF21, FGF22, FGF23.

Differentiation of PSCs into DE culture and subsequently into various intermediate mature intestinal cell types can be determined by the presence of stage-specific cell markers. In some embodiments, expression of representative cellular constituents is used to determine DE formation. The representative cellular constituents include but are not limited to CMKOR1, CXCR4, GPR37, RTN4RL1, SLC5A9, SLC40A1, TRPA1, AGPAT3, APOA2, C20orf56, C21orf129, CALCR, CCL2, CER1, CMKOR1, CRIP1, CXCR4, CXorf1, DIO3, DIO30S, EB-1, EHHADH, ELOVL2, EPSTI1, FGF17, FLJ10970, FLJ21195, FLJ22471, FLJ23514, FOXA2, FOXQ1, GATA4, GPR37, GSC, LOC283537, MYL7, NPPB, NTN4, PRSS2, RTN4RL1, SEMA3E, SIAT8D, SLC5A9, SLC40A1, SOX17, SPOCK3, TMOD1, TRPA1, TTN, AW166727, AI821586, BF941609, AI916532, BC034407, N63706 or AW772192, or any combination thereof. In some embodiments, the absence of cellular constituents, such as foregut markers Pdx1 and Albumin, can be used to reveal directed hindgut formation. In some embodiments, one or more (e.g. at least 1,3) intestinal transcription factors CDX2, KLF5 or SOX9 can be used to represent intestinal development. In some embodiments, one or more of GATA4 or GATA6 protein expression can be used to represent intestinal development.

In some embodiments, morphological changes can be used to represent the progress of directed differentiation. In some embodiments, spheroids (e.g., mid-hindgut, hindgut, anterior foregut, or posterior foregut spheroids) are subject to 3-dimensional culture conditions for maturation. In some embodiments, the gastrointestinal organoids mature in a number of days that is, is about, is at least, is at least about, is not more than, or is not more than about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or any number of days within a range defined by any two of the aforementioned number of days, for example, 6 to 60 days, 20 to 50 days, 30 to 40 days, 6 to 50 days, or 30 to 60 days. In some embodiments, a highly convoluted epithelium surrounded by mesenchymal cells can be observed following spheroid formation. In some embodiments, gastrointestinal organoids, polarized columnar epithelium, goblet cells, or smooth muscle cells can be observed in a number of days that is, is about, is at least, is at least about, is not more than, or is not more than about, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days, or any number of days within a range defined by any two of the aforementioned number of days, for example, 6 to 60 days, 20 to 50 days, 30 to 40 days, 6 to 50 days, or 30 to 60 days.

In some embodiments, pluripotent stem cells are converted into intestinal cell types via a “one step” process. For example, one or more molecules that can differentiate pluripotent stem cells into DE culture (e.g., Activin A) are combined with additional molecules that can promote directed differentiation of DE culture (e.g., Wnt3a and FGF4) to directly treat pluripotent stem cells.

In some embodiments, pluripotent stem cells are prepared from somatic cells. In some embodiments, pluripotent stem cells are prepared from biological tissue obtained from a biopsy. In some embodiments, pluripotent stem cells are prepared from PBMCs. In some embodiments, human PSCs are prepared from human PBMCs. In some embodiments, pluripotent stem cells are prepared from cryopreserved PBMCs. In some embodiments, PBMCs are grown on a feeder cell substrate. In some embodiments, PBMCs are grown on a mouse embryonic fibroblast (MEF) feeder cell substrate. In some embodiments, PBMCs are grown on an irradiated MEF feeder cell substrate. In some embodiments, PBMCs are grown on 0.1% gelatin.

In some embodiments, pluripotent stem cells are prepared from PBMCs by viral transduction. In some embodiments, PBMCs are transduced with Sendai virus, lentivirus, adenovirus, or adeno-associated virus, or any combination thereof. In some embodiments, PBMCs are transduced with Sendai virus comprising expression vectors for Oct3/4, Sox2, Klf4, or L-Myc, or any combination thereof. In some embodiments, PBMCs are transduced with one or more viruses at an MOI that is, is about, is at least, is at least about, is not more than, or is not more than about, 0, 0.1, 0.5, 1.0, 1.5, 2.0, 2.5, 3.0, 3.5, 4.0, 4.5, or 5.0 MOI, or any MOI within a range defined by any two of the aforementioned MOIs, for example, 0 to 5.0, 1.0 to 4.0, 2.0 to 3.0, 0 to 3.0, or 1.0 to 5.0. In some embodiments, after transduction, PBMCs express stem cell reprogramming factors. In some embodiments, after transduction, PBMCs are reprogrammed to iPSCs. In some embodiments, iPSCs are grown on a feeder cell substrate. In some embodiments, iPSCs are grown on a MEF feeder cell substrate. In some embodiments, iPSCs are grown on an irradiated MEF feeder cell substrate. In some embodiments, iPSCs are grown on 0.1% gelatin. In some embodiments, iPSCs are grown in RPMI 1640, DMEM, DMEM/F12, Erythroid Expansion Media, Minigut media, StemPro 34 SFM (serum free media), StemPro hESC SFM, mTeSR 1, or mTeSR Plus media.

In some embodiments, reprogrammed iPSCs are expanded in cell culture. In some embodiments, iPSCs are expanded in Matrigel. In some embodiments, iPSCs are expanded in cell culture media comprising a ROCK inhibitor (e.g. Y-27632). In some embodiments, iPSCs are expanded until 80-95% confluence. In some embodiments, the iPSCs are differentiated into definitive endoderm cells. In some embodiments, iPSCs are differentiated into definitive endoderm cells by contacting the iPSCs with Activin A. In some embodiments, the iPSCs are further contacted with BMP4. In some embodiments, the iPSCs are contacted with a concentration of BMP4 that is, is about, is at least, is at least about, is not more than, or is not more than about, 1, 2, 3, 4, 5, 6, 7, 8, 9, 10, 11, 12, 13, 14, or 15 ng/mL of BMP4.

In some embodiments, the definitive endoderm cells are differentiated to mid-hindgut spheroids. In some embodiments, the definitive endoderm cells are differentiated to mid-hindgut spheroids by contacting the definitive endoderm cells with one or more (e.g. at least 1 or 2) of a GSK3 inhibitor or FGF4. In some embodiments, the GSK3 inhibitor is CHIR99021. In some embodiments, the FGF4 is recombinant FGF4. In some embodiments, the definitive endoderm cells are differentiated to mid-hindgut spheroids without contacting the definitive endoderm cells with one or more (e.g. at least 1 or 2) of a GSK3 inhibitor or FGF4. In some embodiments, the definitive endoderm cells are differentiated to mid-hindgut spheroids without contacting the definitive endoderm with CHIR99021 or FGF4, or both. In some embodiments, the definitive endoderm cells are differentiated to mid-hindgut spheroids by contacting the definitive endoderm cells with epidermal growth factor (EGF).

In some embodiments, the mid-hindgut spheroids are embedded in a basement membrane or basement membrane mimetic. In some embodiments, the mid-hindgut spheroids are embedded in Matrigel. In some embodiments, the mid-hindgut spheroids are cultured in basal gut medium. In some embodiments, the mid-hindgut spheroids are cultured in basal gut medium to differentiate the mid-hindgut spheroids to intestinal organoids. In some embodiments, basal gut medium comprises one or more of Advanced DMEM-F12, N2 supplement, B27 supplement without vitamin A, HEPES, L-glutamine, penicillin-streptomycin, or epidermal growth factor (EGF), or any combination thereof. In some embodiments, basal gut medium comprises EGF. In some embodiments, the mid-hindgut spheroids are filtered through a pore. In some embodiments, the mid-hindgut spheroids are filtered through 70 μm pore size. In some embodiments, the mid-hindgut spheroids are separated into spheroids that are smaller than 70 μm and spheroids that are larger than 70 μm. In some embodiments, the spheroids that are larger than 70 μm are used for the methods described herein.

In some embodiments, the definitive endoderm cells are differentiated to spheroids. In some embodiments, the definitive endoderm cells are differentiated to spheroids by contacting the definitive endoderm cells with one or more (e.g. at least 1, 2, 3, 4) of a GSK3 inhibitor, FGF4, BMP inhibitor, or retinoic acid (RA). In some embodiments, the GSK3 inhibitor is CHIR99021. In some embodiments, the FGF4 is recombinant FGF4. In some embodiments, the BMP inhibitor is Noggin. In some embodiments, the definitive endoderm cells are differentiated to spheroids without contacting the definitive endoderm cells with one or more (e.g. at least 1, 2, 3, 4) of a GSK3 inhibitor, FGF4, BMP inhibitor, or RA, or any combination thereof. In some embodiments, the definitive endoderm cells are differentiated to spheroids without contacting the definitive endoderm with CHIR99021, FGF4, Noggin, or RA, or any combination thereof. In some embodiments, the definitive endoderm cells are differentiated to spheroids by contacting the definitive endoderm cells with epidermal growth factor (EGF).

In some embodiments, the spheroids are embedded in a basement membrane or basement membrane mimetic. In some embodiments, the spheroids are embedded in Matrigel. In some embodiments, the spheroids are cultured in a growth medium to differentiate the spheroids to organoids. In some embodiments, the spheroids are filtered through a pore. In some embodiments, the spheroids are filtered through 70 μm pore size. In some embodiments, the spheroids are separated into spheroids that are smaller than 70 μm and spheroids that are larger than 70 μm. In some embodiments, the spheroids that are larger than 70 μm are used for the methods described herein.

### Unshaped Organoids

In some embodiments, the gastrointestinal organoids are esophageal organoids, gastric organoids, fundic gastric organoids, antral gastric organoids, small intestinal (intestinal) organoids, or large intestinal (colonic) organoids. In some embodiments, the gastrointestinal organoids are intestinal organoids. In some embodiments, the gastrointestinal organoids are human intestinal organoids (HIOs). In some embodiments, the gastrointestinal organoids are not formed by any of the methods disclosed herein. In some embodiments, the gastrointestinal organoids comprise a generally spherical three-dimensional structure comprising a polarized, columnar epithelium. In some embodiments, the polarized, columnar epithelium is surrounded by a mesenchyme. In some embodiments, the mesenchyme comprises a smooth muscle-like layer. In some embodiments, the epithelium comprises crypt-like proliferative zones and villus-like structures. In some embodiments, the mesenchyme comprises laminated longitudinal and circular muscle. In some embodiments, the gastrointestinal organoid comprises a lamina propria with all of the major functional cell types of a gastrointestinal organ. In some embodiments, the generally spherical gastrointestinal organoid comprises a stratified mesenchyme.

### Shaped Organoids and Methods of Making the Same

In some embodiments, the gastrointestinal organoids are shaped gastrointestinal organoids. In some embodiments, the gastrointestinal organoids are shaped esophageal organoids, shaped gastric organoids, shaped fundic gastric organoids, shaped antral gastric organoids, shaped small intestinal (intestinal) organoids, or shaped large intestinal (colonic) organoids, or any combination thereof. In some embodiments, the gastrointestinal organoids are intestinal organoids. In some embodiments, the shaped gastrointestinal organoids are HIOs. In some embodiments, the shaped gastrointestinal organoids comprise a generally tubular three-dimensional structure comprising a polarized, columnar epithelium. In some embodiments, the polarized, columnar epithelium is surrounded by a mesenchyme. In some embodiments, the mesenchyme comprises a smooth muscle-like layer. In some embodiments, the epithelium comprises crypt-like proliferative zones and villus-like structures. In some embodiments, the mesenchyme comprises laminated longitudinal and circular muscle. In some embodiments, the shaped gastrointestinal organoid comprises a lamina propria with all of the major functional cell types of a gastrointestinal organ. In some embodiments, the shaped gastrointestinal organoid comprises a stratified mesenchyme.

In some embodiments, the shaped gastrointestinal organoids are elongated gastrointestinal organoids. In some embodiments, the gastrointestinal organoids are formed into an elongated structure. In some embodiments, the gastrointestinal organoids are formed into an elongated structure using one of the formation tray embodiments described herein. In some embodiments, the shaped gastrointestinal organoids have a straight or essentially straight shape. In some embodiments, the shaped gastrointestinal organoids have a shape where at least one dimension is straight or essentially straight. In some embodiments, the shaped gastrointestinal organoids have a shape where all dimensions are straight or essentially straight. In some embodiments, the shaped gastrointestinal organoids have a cuboid, cubic, cylindrical, conical, or pyramidal shape. In some embodiments, the shaped gastrointestinal organoids have a curved or essentially curved shape. In some embodiments, the shaped gastrointestinal organoids have a shape where at least one dimension is curved or essentially curved. In some embodiments, the shaped gastrointestinal organoids have a shape where all dimensions are curved or essentially curved. In some embodiments, the shaped gastrointestinal organoids have a spherical shape. In some embodiments, the shaped gastrointestinal organoids have a non-spherical shape. In some embodiments, the shaped gastrointestinal organoids have a shape that has at least one curved surface but would otherwise be straight. In some embodiments, the shaped gastrointestinal organoids have a curved cuboid, curved cubic, curved cylindrical, curved conical, curved pyramidal, parabolic, paraboloidal, hyperbolic, hyperboloidal, ellipsoidal, spiral, helical, sine wave, sinusoidal, serpentine, square wave, triangle wave, sawtooth wave, fusiform, dendritic, branching, or radial shape, or any combination thereof. In some embodiments, spheroids (e.g. mid-hindgut spheroids) prepared as disclosed herein are seeded into a groove of a collection channel of a formation tray. In some embodiments, spheroids are seeded into a collection channel at a number of spheroids that is, is about, is at least, is at least about, is not more than, or is not more than about, 100, 500, 1000, 1500, 2000, 2500, 3000, 3500, 4000, 4500, 5000, 5500, 6000, 6500, 7000, 7500, 8000, 8500, 9000, or 10000 spheroids, or any number of spheroids within a range defined by any two of the aforementioned numbers, for example, 100 to 10000 spheroids, 2000 to 8000 spheroids, 3000 to 4000 spheroids, 100 to 4000 spheroids, or 3000 to 10000 spheroids, per collection channel. In some embodiments, the spheroids are seeded into a collection channel at a density that is, is about, is at least, is at least about, is not more than, or is not more than about, 100, 200, 300, 400, 500, 600, 700, 800, 900, 1000, 1100, 1200, 1300, 1400, 1500, 1600, 1700, 1800, 1900, or 2000 spheroids per mm³, or any density within a range defined by any two of the aforementioned densities, for example 100 to 2000 spheroids per mm³, 500 to 1500 spheroids per mm³, 100 to 1000 spheroids per mm³, or 1000 to 2000 spheroids per mm³. In some embodiments, the spheroids in the collection channel of the formation tray or in the Tissue Train Culture plate, or both, are cultured in Minigut media, which comprises one or more of Advanced DMEM-F12, glutamine, HEPES, penicillin, streptomycin, N2 supplement, B27 supplement or EGF, or any combination thereof. In some embodiments, the Minigut media comprises EGF. In some embodiments, the EGF is recombinant EGF. In some embodiments, the spheroids in the collection channel of the formation tray or in the Tissue Train Culture Plate, or both, are cultured in Matrigel. In some embodiments, the spheroids in the collection channel of the formation tray or in the Tissue Train Culture Plate, or both, are cultured in a 50% mixture of Matrigel and Minigut media. In some embodiments, the spheroids are grown in the collection channel for a number of days that is, is about, is at least, is at least about, is not more than, or is not more than about, 1, 2, 3, 4, 5, 6, 7, 8, 9, or 10 days, or a range of any two of the preceding values, for example 1-10 days, 3-7 days, 1-5 days, 4-10 days, 6-9 days, or 7-10 days. The type of spheroid selected is determined by the desired organoid, for example, mid-hindgut spheroids for the preparation of intestinal organoids, hindgut spheroids for the preparation of colonic organoids, anterior foregut spheroids for the preparation of esophageal organoids, or posterior foregut spheroids for the preparation of gastric organoids. In some embodiments, the spheroids are mid-hindgut spheroids. In some embodiments, the spheroids are hindgut spheroids. In some embodiments, the spheroids are foregut spheroids. In some embodiments, the spheroids are anterior foregut spheroids. In some embodiments, the spheroids are posterior foregut spheroids.

In some embodiments, the shaped gastrointestinal organoid is an elongated gastrointestinal organoid. In some embodiments, the elongated gastrointestinal organoid comprises an elongate length, a width, a depth, or a diameter, or any combination thereof. In some embodiments, the elongate length is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 millimeters, or any length within a range defined by any two of the aforementioned lengths, for example, 1 to 50 mm, 10 to 40 mm, 20 to 30 mm, 1 to 30 mm, or 20 to 50 mm. In some embodiments, the width is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any width within a range defined by any two of the aforementioned widths, for example, 0.2 μm to 3000 μm, 200 μm to 1500 μm, 500 μm to 1000 μm, 0.2 μm to 1000 μm, or 500 μm to 3000 μm. In some embodiments, the depth is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any depth within a range defined by any two of the aforementioned depths, for example, 0.2 μm to 3000 μm, 200 μm to 1500 μm, 500 μm to 1000 μm, 0.2 μm to 1000 μm, or 500 μm to 3000 μm. In some embodiments, the diameter is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any diameter within a range defined by any two of the aforementioned diameters, for example, 0.2 μm to 3000 μm, 200 μm to 1500 μm, 500 μm to 1000 μm, 0.2 μm to 1000 μm, or 500 μm to 3000 μm. In some embodiments, the ratio of the elongate length to one or more (e.g. 1, 2, 3) of the width, depth, or diameter, or any combination thereof is, is about, is at least, is at least about, is not more than, or is not more than about 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, 10000, 20000, 30000, 40000, 500000, 60000, 70000, 80000, 90000, 100000, 200000, 300000, 400000, or 500000, or any ratio between a range defined by any two of the aforementioned ratios, for example, 1 to 500000, 100 to 500000, 1000 to 10000, 1 to 500000, or 1000 to 500000. In some embodiments, the volume of the elongated gastrointestinal organoid is, is about, is at least, is at least about, is not more than, or is not more than about 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or 0.01 mm³, 0.1 mm³, 1 mm³, 2 mm³, 3 mm³, 4 mm³, 5 mm³, 6 mm³, 7 mm³, 8 mm³, 9 mm³, 10 mm³, 100 mm³, 1000 mm³, 1500 mm³, or 2000 mm³, or any volume within a range defined by any two of the aforementioned volumes, for example, 100 μm³ to 2000 mm³, 1000 μm³ to 1000 mm³, 0.1 mm³ to 5 mm³, 100 μm³ to 1 mm³, or 1 mm³ to 2000 mm³. In some embodiments, the elongated gastrointestinal spheroid is comprised by or formed from a number of spheroids that is, is about, is at least, is at least about, is not more than, or is not more than about, 100, 500, 1000, 1500, 2000, 2500, 3000, 3500, 4000, 4500, 5000, 5500, 6000, 6500, 7000, 7500, 8000, 8500, 9000, or 10000 spheroids, or any number of spheroids within a range defined by any two of the aforementioned numbers, for example, 100 to 10000 spheroids, 2000 to 8000 spheroids, 3000 to 4000 spheroids, 100 to 4000 spheroids, or 3000 to 10000 spheroids. In some embodiments, the elongated gastrointestinal spheroid is comprised by or formed from spheroids that are gathered at a density that is, is about, is at least, is at least about, is not more than, or is not more than about, 100, 200, 300, 400, 500, 600, 700, 800, 900, 1000, 1100, 1200, 1300, 1400, 1500, 1600, 1700, 1800, 1900, or 2000 spheroids per mm³, or any density within a range defined by any two of the aforementioned densities, for example 100 to 2000 spheroids per mm³, 500 to 1500 spheroids per mm³, 100 to 1000 spheroids per mm³, or 1000 to 2000 spheroids per mm³. As mentioned previously, the type of spheroid selected is determined by the desired organoid, for example, mid-hindgut spheroids for the preparation of intestinal organoids, hindgut spheroids for the preparation of colonic organoids, anterior foregut spheroids for the preparation of esophageal organoids, or posterior foregut spheroids for the preparation of gastric organoids. In some embodiments, the spheroids are mid-hindgut spheroids. In some embodiments, the spheroids are hindgut spheroids. In some embodiments, the spheroids are foregut spheroids. In some embodiments, the spheroids are anterior foregut spheroids. In some embodiments, the spheroids are posterior foregut spheroids.

In some embodiments, the spheroids are subjected to tension while formed in the collection channel. In some embodiments, the spheroids are subjected to tension after being formed in the collection channel. In some embodiments, spheroids formed in a collection channel is grown in a Tissue Train Culture Plate as described in more detail herein. In some embodiments, the Tissue Train Culture Plate comprises nylon mesh tabs and a deformable rubber membrane. In some embodiments, the spheroids in the shaped form are aligned between nylon mesh tabs and anchored to the nylon mesh tabs. In some embodiments, the deformable rubber membrane imparts a mechanical load on the formed spheroids. In some embodiments, the deformable rubber membrane imparts a uniaxial strain on the formed spheroids. In some embodiments, the deformable rubber membrane imparts a uniaxial strain that induces a percent elongation that is, is about, is at least, is at least about, is not more than, or is not more than about, 0%, 1%, 2%, 3%, 4%, 5%, 6%, 7%, 8%, 9%, 10%, 11%, 12%, 13%, 14%, 15%, 16%, 17%, 18%, 19%, or 20% elongation, or any percentage within a range defined by any two of the aforementioned percentages, for example, 0% to 20%, 5% to 15%, 8% to 12%, 0% to 10%, or 10% to 20%. In some embodiments, the uniaxial strain enhances an elongated structure for the spheroids. In some embodiments, spheroids experiencing the uniaxial strain differentiate into elongated gastrointestinal organoids. The type of spheroid selected is determined by the desired organoid, for example, mid-hindgut spheroids for the preparation of intestinal organoids, hindgut spheroids for the preparation of colonic organoids, anterior foregut spheroids for the preparation of esophageal organoids, or posterior foregut spheroids for the preparation of gastric organoids. In some embodiments, the spheroids are mid-hindgut spheroids. In some embodiments, the spheroids are hindgut spheroids. In some embodiments, the spheroids are foregut spheroids. In some embodiments, the spheroids are anterior foregut spheroids. In some embodiments, the spheroids are posterior foregut spheroids.

In some embodiments, the gastrointestinal organoids described herein are esophageal organoids, gastric organoids, fundic gastric organoids, antral gastric organoids, intestinal organoids, or colonic organoids, or any combination thereof. In some embodiments, the gastrointestinal organoids described herein are intestinal organoids. In some embodiments, the gastrointestinal organoids described herein are HIOs. In some embodiments, the intestinal organoids are produced according to the methods described herein. In some embodiments, the gastrointestinal organoids are produced according to the methods described herein. In some embodiments, unshaped gastrointestinal organoids are produced according to methods known in the art. In some embodiments, spheroids (e.g. mid-hindgut, hindgut, anterior foregut, or posterior foregut spheroids) and unshaped gastrointestinal organoids (e.g. esophageal, gastric, fundic gastric, antral gastric, intestinal, or colonic organoids) and methods of making the same have been described in U.S. Pat. Nos. 9,719,068 and 10,174,289, and PCT Publications WO 2016/061464, WO 2017/192997, WO 2018/106628, WO 2019/074793, each of which are hereby expressly incorporated by reference for the purposes of producing respective unshaped gastrointestinal organoids. In some embodiments, the gastrointestinal organoids described herein, or unshaped gastrointestinal organoids described in the referenced publications are prepared as shaped gastrointestinal organoids using one or more formation trays described herein. In some embodiments, the spheroids (e.g. mid-hindgut, hindgut, anterior foregut, or posterior foregut spheroids) described herein or described in the referenced publications are used to prepare shaped gastrointestinal organoids (e.g. esophageal, gastric, fundic gastric, antral gastric, intestinal, or colonic organoids) by culturing the spheroids in one or more collection channels of a formation tray described herein to differentiate the spheroids into the shaped gastrointestinal organoids. In some embodiments, the culturing of the spheroids in the collection channels is under conditions disclosed in the referenced publications for the particular organoid of interest. In some embodiments, the spheroids described herein or described in the referenced publications are used to prepare shaped intestinal organoids. In some embodiments, the spheroids described herein or described in the referenced publications are used to prepare shaped HIOs. In some embodiments, one or more formation trays described herein are used to form shaped gastrointestinal organoids (e.g. esophageal, gastric, fundic gastric, antral gastric, intestinal, or colonic organoids) comprising one or more features described herein. In some embodiments, one or more formation trays described herein are used to form shaped intestinal organoids In some embodiments, one or more formation trays described herein are used to form shaped HIOs.

### Formation Tray Embodiments

Disclosed herein are embodiments of a formation tray that is used to prepare shaped organoids structures from a plurality of spheroids. In some embodiments, the shaped organoid structures are for use in, for example, investigating gastrointestinal function or transplant purposes into a host organism (e.g. a human, mouse, rat, dog, cat, or other mammal). In some embodiments, the shaped organoid structures are elongated organoid structures. In some embodiments the spheroids are mid-hindgut spheroids and the elongated organoids are intestinal organoids, for example elongated HIOs. In some embodiments, the formation tray (10) has a structure designed for a predetermined shape configured to more closely complement a desired organ for use. The formation tray (10) more particularly has one or more (e.g. at least 1, 3, 5, 10) collection channels (12) configured to receive the spheroids and gather the spheroids according to the collective arrangement defining the predetermined shape. Continued culturing of the spheroids for a predetermined period of formation time within the one or more collection channel (12) effectively assigns the spheroids relative to each other in a cast state configured to maintain the spheroids in the predetermined shape, particularly upon removal from the one or more collection channels (12) for further culturing and/or implantation. In some embodiments, the predetermined shape is a non-spherical predetermined shape, such as an elongate column. In some embodiments, the elongate column predetermined shape of the one or more collection channels (12) define the cast state to maintain the arrangement of spheroids in the elongate column predetermined shape. In some embodiments, the term “cast state” as used herein has its plain and ordinary meaning in light of the specification and refers to the spheroids being secured relative to each other so as to maintain the predetermined shape while allowing for some movement such that the predetermined shape remains flexible, including but not limited to resilient flexibility or malleable flexibility. In some embodiments, the cast state and predetermined shape are not intended to limit the arrangement of spheroids to a rigid, fixed state, and it will be appreciated that the predetermined shape will be sufficiently maintained for complementing the desired organ functionality while allowing for manipulation and structural connection to the desired organ by a surgeon during implantation.

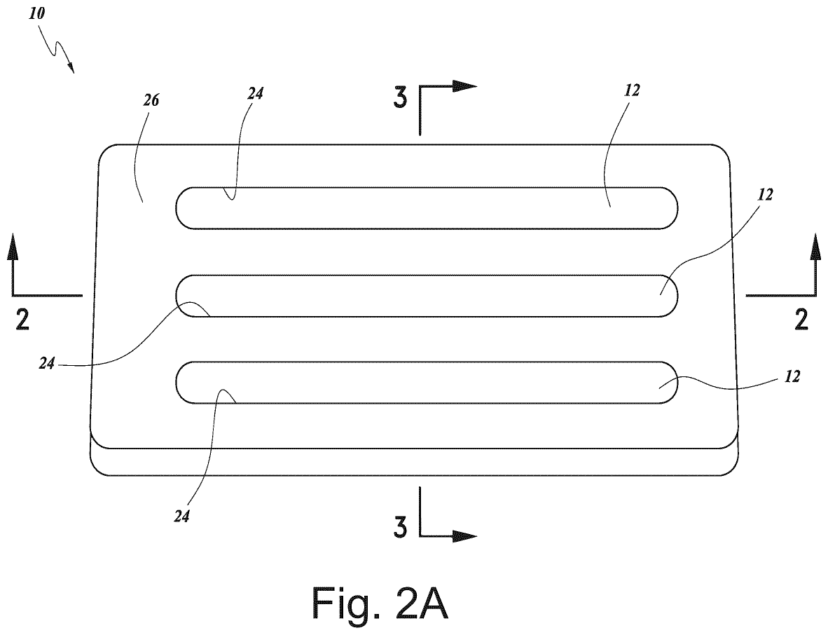

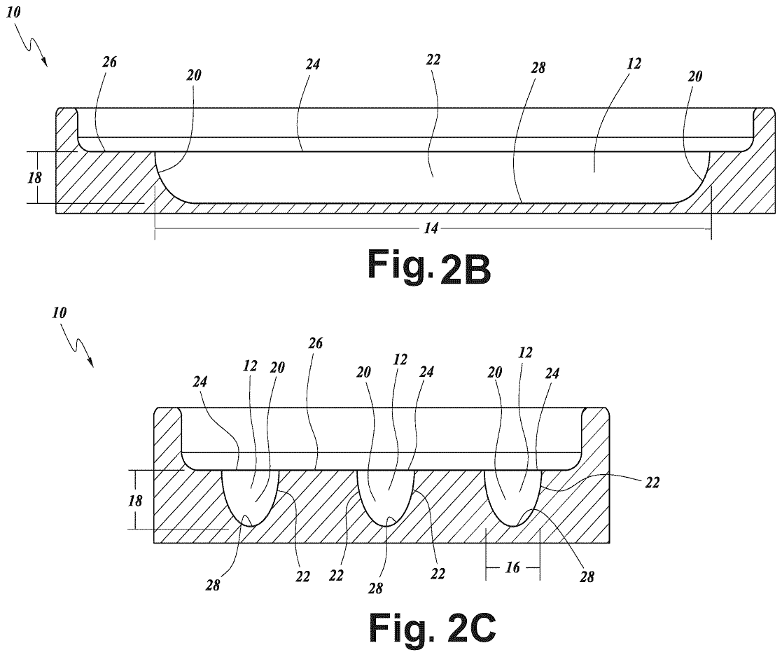

FIGS. 2A-C illustrate an embodiment of the formation tray (10) comprising a plurality of collection channels (12). Each of plurality of collection channels (12) of the present embodiment has an elongate length (14), a width (16), and a depth (18). In some embodiments, the elongate length (14) extends in a longitudinal direction that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 millimeters, or any length within a range defined by any two of the aforementioned lengths, for example, 1 to 50 mm, 10 to 40 mm, 20 to 30 mm, 1 to 30 mm, or 20 to 50 mm, and is defined by opposing longitudinal sidewalls (20) of the formation tray (10). In some embodiments, the width (16) extends in a lateral direction that is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any width within a range defined by any two of the aforementioned widths, for example, 0.2 μm to 3000 μm, 200 μm to 1500 μm, 500 μm to 1000 μm, 0.2 μm to 1000 μm, or 500 μm to 3000 μm, perpendicular to the longitudinal direction, and is defined by opposing lateral sidewalls (22). In some embodiments, the depth (18) extends in a transverse direction that is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any depth within a range defined by any two of the aforementioned depths, for example, 0.2 μm to 3000 μm, 200 μm to 1500 μm, 500 μm to 1000 μm, 0.2 μm to 1000 μm, or 500 μm to 3000 μm, perpendicular to the longitudinal and lateral directions. In some embodiments, the depth (18) is defined between a channel opening (24) in an upper surface (26) of the formation tray (10) and a floor surface (28) of the formation tray (10). In some embodiments, each of the plurality of collection channels (12) is thus defined between respective longitudinal sidewalls (20), lateral sidewalls (22), the channel opening (24), and the floor surface (28). In some embodiments, each of the plurality of collection channels (12) has hemispherical longitudinal end portions with a radius of curvature that is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any radius within a range defined by any two of the aforementioned radii, for example, 0.2 μm to 3000 μm, 200 μm to 1500 μm, 500 μm to 1000 μm, 0.2 μm to 1000 μm, or 500 μm to 3000 μm, with a generally cylindrical shape extending therebetween with another radius of curvature that is, is about, is at least, is at least about, is not more than, or is not more than about 0.2 μm, 1 μm, 5 μm, 10 μm, 50 μm, 100 μm, 200 μm, 300 μm, 400 μm, 500 μm, 600 μm, 700 μm, 800 μm, 900 μm, 1000 μm, 1200 μm, 1300 μm, 1400 μm, 1500 μm, 1600 μm, 1700 μm, 1800 μm, 1900 μm, 2000 μm, 2500 μm, or 3000 μm, or any radius within a range defined by any two of the aforementioned radii, for example, 0.2 μm to 3000 μm, 200 μm to 1500 μm, 500 μm to 1000 μm, 0.2 μm to 1000 μm, or 500 μm to 3000 μm. In some embodiments, each of the plurality of collection channels (12) has a volume that is, is about, is at least, is at least about, is not more than, or is not more than about 100 μm³, 200 μm³, 300 μm³, 400 μm³, 500 μm³, 600 μm³, 700 μm³, 800 μm³, 900 μm³, 1000 μm³, 10000 μm³, 100000 μm³, 1000000 μm³, or 0.01 mm³, 0.1 mm³, 1 mm³, 2 mm³, 3 mm³, 4 mm³, 5 mm³, 6 mm³, 7 mm³, 8 mm³, 9 mm³, 10 mm³, 100 mm³, 1000 mm³, 1500 mm³, or 2000 mm³, or any volume within a range defined by any two of the aforementioned volumes, for example, 100 μm³ to 2000 mm³, 1000 μm³ to 1000 mm³, 0.1 mm³ to 5 mm³, 100 μm³ to 1 mm³, or 1 mm³ to 2000 mm³. In some embodiments, the formation tray (10) has a plurality of collection channels (12) or one or more (e.g. at least 1, 2, 3, 4, 5, 6, 7, 8, 9, 10) collection channels (12) with the same or about the same length (14), the same or about the same width (16), and the same or about the same depth (18) dimension. In some embodiments, the collection channels of the plurality of collection channels (12) or one or more collection channels (12) do not necessarily have the same or about the same length (14), do not necessarily have the same or about the same width (16), or do not necessarily have the same or about the same depth (18) dimension, or any combination thereof. In some embodiments, the formation tray (10) has a plurality of collection channels (12) or one or more collection channels (12) that are parallel or about parallel to each other. In some embodiments, the collection channels of the plurality of collection channels (12) or one or more collection channels (12) are not necessarily parallel or about parallel to each other. In some embodiments, the formation tray (10) comprises a lid configured to cover one or more (e.g. at least 1, 2, 3, 4, 5, 6, 7, 8, 9, 10) of the collection channels of a plurality of collection channels (12) or the one or more collection channels (12). In some embodiments, one or more collection channels (12) are formed without a channel opening (24) so as to be encapsulated rather than open at the upper surface (26). In some embodiments, the one or more (e.g. at least 1, 2, 3, 4, 5, 6, 7, 8, 9, 10) encapsulated collection channels (12) comprise a tube structure or a hose structure. In some embodiments, the formation tray (10) is not intended to be unnecessarily limited to the particular number, arrangement, or size collection channels (12) shown in the embodiments of FIGS. 2A-C or described herein.

In some embodiments, the one or more collection channels (12) are configured to gather the spheroids together towards the collective arrangement defining the predetermined shape. In some embodiments, the one or more collection channels (12) taper together from the relatively wider channel opening (24) towards a relatively narrower floor surface (28). FIGS. 2A-C illustrate an embodiment of a formation tray (10) where opposing longitudinal sidewalls (20) taper toward each other from the channel opening (24) to the floor surface (28), while the opposing lateral sidewalls (22) similarly taper toward each other from the channel opening (24) to the floor surface (28). In some embodiments, gravity forces the spheroids in the one or more collection channels (12) downward in the transverse direction while the reactionary forces applied to the spheroids by the longitudinal and lateral sidewalls (20, 22), direct the spheroids upward and inward toward each other to effectively gather the spheroids together in the predetermined shape.

In some embodiments, the one or more collection channels (12) are not limited by the embodiments depicted in FIGS. 2A-C. In some embodiments, the one or more collection channels have a straight or essentially straight shape. In some embodiments, the one or more collection channels have a shape where at least one dimension is straight or essentially straight. In some embodiments, the one or more collection channels have a shape where all dimensions are straight or essentially straight. In some embodiments, the one or more collection channels have a cuboid, cubic, cylindrical, conical, or pyramidal shape. In some embodiments, the one or more collection channels have a curved or essentially curved shape. In some embodiments, the one or more collection channels have a shape where at least one dimension is curved or essentially curved. In some embodiments, the one or more collection channels have a shape where all dimensions are curved or essentially curved. In some embodiments, the one or more collection channels have a spherical shape. In some embodiments, the one or more collection channels have a non-spherical shape. In some embodiments, the one or more collection channels have a shape that has at least one curved surface but would otherwise be straight. In some embodiments, the one or more collection channels have a curved cuboid, curved cubic, curved cylindrical, curved conical, curved pyramidal, parabolic, paraboloidal, hyperbolic, hyperboloidal, ellipsoidal, spiral, helical, sine wave, sinusoidal, serpentine, square wave, triangle wave, sawtooth wave, fusiform, dendritic, branching, or radial shape, or any combination thereof. In some embodiments, the shaped gastrointestinal organoid properly forms in any one of the shapes of the one or more collection channels described herein and elsewhere.

In some embodiments, each of the one or more collection channels (12) comprise a number of spheroids and liquid media appropriate for the volume of the one or more collection channels (12). In some embodiments, each of the one or more collection channels (12) comprise a number of spheroids that is, is about, is at least, is at least about, is not more than, or is not more than about, 100, 500, 1000, 1500, 2000, 2500, 3000, 3500, 4000, 4500, 5000, 5500, 6000, 6500, 7000, 7500, 8000, 8500, 9000, or 10000 spheroids, or any number of spheroids within a range defined by any two of the aforementioned numbers, for example, 100 to 10000 spheroids, 2000 to 8000 spheroids, 3000 to 4000 spheroids, 100 to 4000 spheroids, or 3000 to 10000 spheroids, per collection channel (12). In some embodiments, each of the one or more collection channels (12) that comprise a number of spheroids that is, is about, is at least, is at least about, is not more than, or is not more than about, 100, 500, 1000, 1500, 2000, 2500, 3000, 3500, 4000, 4500, 5000, 5500, 6000, 6500, 7000, 7500, 8000, 8500, 9000, or 10000 spheroids, or any number of spheroids within a range defined by any two of the aforementioned numbers, for example, 100 to 10000 spheroids, 2000 to 8000 spheroids, 3000 to 4000 spheroids, 100 to 4000 spheroids, or 3000 to 10000 spheroids, has a length (14) that is, is about, is at least, is at least about, is not more than, or is not more than about, 10, 15 or 20 mm, a width (16) that is, is about, is at least, is at least about, is not more than, or is not more than about, 0.5 mm, and a depth (18) that is, is about, is at least, is at least about, is not more than, or is not more than about, 0.5 mm. In some embodiments, the spheroids gather with a predetermined density that is, is about, is at least, is at least about, is not more than, or is not more than about, 100, 200, 300, 400, 500, 600, 700, 800, 900, 1000, 1100, 1200, 1300, 1400, 1500, 1600, 1700, 1800, 1900, or 2000 spheroids per mm³, or any density within a range defined by any two of the aforementioned densities, for example 100 to 2000 spheroids per mm³, 500 to 1500 spheroids per mm³, 100 to 1000 spheroids per mm³, or 1000 to 2000 spheroids per mm³.

In the embodiments illustrated in FIGS. 2A-C, the longitudinal sidewalls (20), the lateral sidewalls (22), and the floor surface (28) of the one or more collection channels (12) are arcuate, each having a radius of curvature as to be tubular and continuous with each other. In some embodiments, one or more (e.g. at least 1, 3, 5, 10) of the longitudinal sidewalls (20), the lateral sidewalls (22), or the floor surface (28) intersect so as to not be continuous. In some embodiments, one or more (e.g. at least 1, 3, 5, 10) sidewalls (20, 22) and one or more (e.g. at least 1, 3, 5, 10) floor surfaces (28) of the one or more collection channels (12) are therefore not intended to be unnecessarily limited to the smooth, continuous surfaces shown in the embodiments of FIGS. 2A-C or described herein. In some embodiments, the shapes and dimensions of the one or more (e.g. at least 1, 3, 5, 10) collection channels (12) are configured for effective growth of spheroids such as gastrointestinal spheroids described herein. In some embodiments, the shapes and dimension of the one or more (e.g. at least 1, 3, 5, 10) collection channels (12) are configured for effective growth of a spheroid that is not a gastrointestinal spheroid. In some embodiments, the one or more collection channels (12) are not intended to be unnecessarily limited to the particular shape and/or dimensions shown in the embodiments of the Figures or described herein.

In some embodiments, the formation tray (10) has a single, unitary structure. In some embodiments, the formation tray (10) is manufactured from a biocompatible material. In some embodiments, the formation tray (10) is manufactured from a biocompatible material that inhibits attachment of the spheroids to the formation tray (10) within the one or more collection channels (12) while allowing for development of the spheroids to the gastrointestinal organoid structures. In some embodiments, the formation tray (10) is formed from a plurality of components wherein at least the surfaces within the one or more collection channels (12) is manufactured from a biocompatible material. In some embodiments, the biocompatible material comprises, consists essentially of, or consists of stainless steel, titanium, a polymeric organosilicon compound, polydimethylsiloxane (PDMS), glass, plastic, PVC, PE, PP, PMMA, PS, PTFE, nylon, polyurethane, PET, PES, hyaluronans, chitosan, sugars, ceramics, alumina, zirconia, bioglass, hydroxyapatite, or any combination thereof, or any other biocompatible material known in the art. In some embodiments, the formation tray (10) is sterile, resistant to adherence by tissues and/or cells, comprises a hydrophobic surface, comprises a feature that improves formation of the disclosed tissues and subsequent removal and/or use, or any combination thereof. In some embodiments, the formation tray (10) comprises one or more (e.g. at least 1, 3, 5, 10) small molecule compounds, activators, inhibitors, growth factors, nucleic acids, DNA, RNA, peptides, polypeptides, or proteins, or any combination thereof, that promotes growth and/or differentiation.

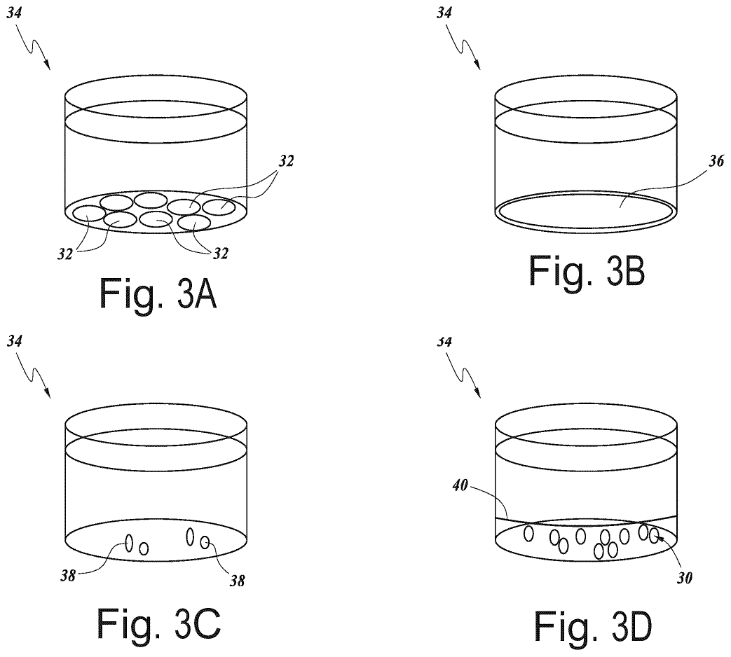

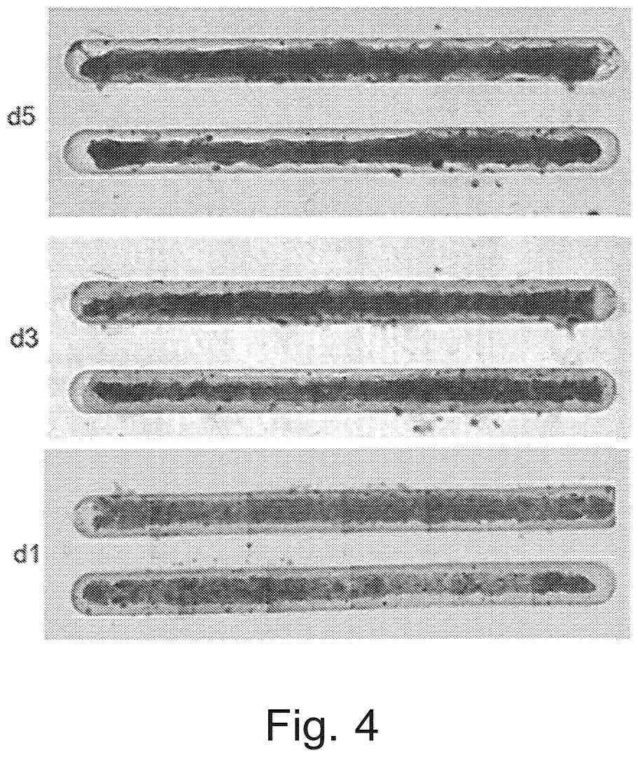

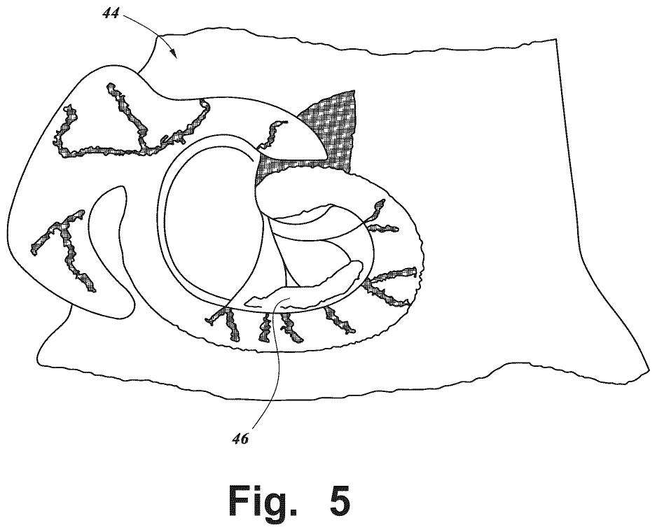

FIGS. 3A-D show an embodiment of a plurality of pre-arranged spheroids (30) for culturing in a formation tray (10). In some embodiments, a plurality of iPSCs (32) is cultured within a biocompatible container (34) under conditions that differentiate the plurality of iPSCs to a plurality of definitive endoderm cells (36), such as the conditions described herein or otherwise known in the art. In some embodiments, the plurality of definitive endoderm cells (36) are cultured under conditions that differentiate the plurality of definitive endoderm cells into a plurality of spheroids (38) within the biocompatible container (34), such as the conditions described herein or otherwise known in the art. In some embodiments, the spheroids (38) are hindgut spheroids. In some embodiments, the spheroids (38) are foregut spheroids. In some embodiments, the spheroids (38) are anterior foregut spheroids. In some embodiments, the spheroids (38) are posterior foregut spheroids. In some embodiments, the spheroids (38) are mid-hindgut spheroids. In some embodiments, the spheroids (38) are not mid-hindgut spheroids. In some embodiments, as the plurality of spheroids (38) are forming, a scaffold strand (40) is introduced proximate to the plurality of spheroids (38). In some embodiments, the scaffold strand (40) is permanently or semi-permanently placed or housed within or close to the biocompatible container (34) such that the spheroids are able to contact the scaffold strand (40) upon forming. In some embodiments, the scaffold strand (40) is formed from a biocompatible material configured to attract and contact developing spheroids (38) and, in turn, urge the developing spheroids (38) into a plurality of pre-arranged spheroids (30). In some embodiments, the scaffold strand (40) has a complementary shape to the collection channel (12) such that the plurality of pre-arranged spheroids (30) are more efficiently collected within the biocompatible container (34) and removed from the biocompatible container (34). In some embodiments, the scaffold strand (40) is generally linear and fibrous. In some embodiments, the scaffold strand (40) is a string, fiber, wire, cable, or other structure configured to attract and arrange the spheroids (38). In some embodiments, the scaffold strand (40) is constructed from a suitable metallic or non-metallic biocompatible material configured to attract the spheroids while allowing for the development of the spheroids to the organoid.

In some embodiments, once the plurality of pre-arranged spheroids (30) are sufficiently seed filtered, the scaffold strand (40) with the pre-arranged spheroids (30) attached thereto are removed and the pre-arranged spheroids (30) are transferred into the one or more collection channels (12) (FIG. 4). In some embodiments, the scaffold strand (40) may then be discarded, leaving the pre-arranged spheroids (30) in the predetermined shape. In some embodiments, while the linearly pre-arranged spheroids (30) simplify placement into the complementary shaped collection channels (12), such spheroids (38) may be cultured and removed from the biocompatible container (34) without the use of the scaffold strand (40). FIG. 4 shows an embodiment of pre-arranged spheroids (30) cultured within the one or more collection channels (12) on day one (d1), day three (d3), and day five (d5). In some embodiments, the pre-arranged spheroids (30) are cultured in the one or more collection channels (12) for a predetermined period of formation time as described herein (see, e.g. above) such that fusion occurs between a mesenchyme of the pre-arranged spheroids (30), a blood supply forms, innervation occurs, or the spheroids adopt the predetermined shape (e.g. an elongate column for an elongated gastrointestinal organoid as described herein), or any combination thereof.

In some embodiments, the pre-arranged spheroids are transferred to a Tissue Train Culture Plate (Flexcell International Corp., Burlington NC). In some embodiments, the Tissue Train Culture Plate comprises nylon mesh tabs and a deformable rubber membrane situated between the nylon mesh tabs. In some embodiments, the pre-arranged spheroids are aligned on the deformable rubber membrane between the nylon mesh tabs such that the length (i.e. longest dimension) of the pre-arranged spheroids are situated on and between the nylon mesh tabs. In some embodiments, the nylon mesh tabs serve as anchors to retain the ends of the pre-arranged spheroids. In some embodiments, the Tissue Train Culture Plate comprises a vacuum chamber underneath the deformable rubber membrane, wherein application of a vacuum to the Tissue Train Culture Plate, the deformable rubber membrane is stretched towards the vacuum chamber. In some embodiments, the pre-arranged spheroids are placed onto the deformable rubber membrane between the nylon mesh tabs while a vacuum is applied to the Tissue Train Culture Plate. In some embodiments, the vacuum is then relieved, returning the deformable rubber membrane to an unstretched state. In some embodiments, the return to the unstretched state imparts a strain on the pre-arranged spheroids that are situated on top of the deformable rubber membrane. In some embodiments, the strain is a uniaxial strain. In some embodiments, the strain is a uniaxial strain directed outwards towards the nylon mesh tabs. In some embodiments, the uniaxial strain imparts a percent elongation onto the pre-arranged spheroids is, is about, is at least, is at least about, is not more than, or is not more than about, 0%, 1%, 2%, 3%, 4%, 5%, 6%, 7%, 8%, 9%, 10%, 11%, 12%, 13%, 14%, 15%, 16%, 17%, 18%, 19%, or 20% elongation, or any percentage within a range defined by any two of the aforementioned percentages, for example, 0% to 20%, 5% to 15%, 8% to 12%, 0% to 10%, or 10% to 20%. In some embodiments, the uniaxial strain imparted by the deformable rubber membrane keeps the pre-arranged spheroids in an elongated shape. In some embodiments, the pre-arranged spheroids are cultured under strain in the Tissue Train Culture Plate for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days of culture within a range defined by any two of the aforementioned days, for example, 1 to 50 days, 10 to 40 days, 20 to 30 days, 1 to 30 days, or 20 to 50 days.

In some embodiments, the size and dimension of the one or more collection channels (12) and/or the size and dimension of the shaped gastrointestinal organoid is appropriately configured for a mouse or other organism that is approximately the size of a mouse. In some embodiments, the size and dimension of the one or more collection channels (12) and/or the size and dimension of the shaped gastrointestinal organoid is appropriately configured for a human. In some embodiments, the elongate length (14) of the collection channel (12) or the length of the shaped gastrointestinal organoid extends in a longitudinal direction that is, is about, is at least, is at least about, is not more than, or is not more than about, 1, 5, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, or 600 cm, or any length within a range defined by any two of the aforementioned lengths, for example, 1 to 600 cm, 100 to 500 cm, 200 to 300 cm, 1 to 300 cm, or 200 to 600 cm. In some embodiments, the width (16) of the collection channel (12) or the width of the shaped gastrointestinal organoid extends in a lateral direction that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm, or any width within a range defined by any two of the aforementioned widths, for example, 1 to 30 cm, 5 to 25 cm, 10 to 20 cm, 1 to 20 cm, or 10 to 30 cm. In some embodiments, the depth (18) of the collection channel (12) or the depth of the shaped gastrointestinal organoid extends in a transverse direction that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm, or any depth within a range defined by any two of the aforementioned depths, for example, 1 to 30 cm, 5 to 25 cm, 10 to 20 cm, 1 to 20 cm, or 10 to 30 cm. In some embodiments, the diameter of the shaped gastrointestinal organoid is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 cm, or any diameter within a range defined by any two of the aforementioned diameters, for example, 1 to 30 cm, 5 to 25 cm, 10 to 20 cm, 1 to 20 cm, or 10 to 30 cm. In some embodiments, the collection channel (12) or the shaped gastrointestinal organoid has a volume that is, is about, is at least, is at least about, is not more than, or is not more than about, 1, 10, 100, 200, 300, 400, 500, 600, 700, 800, 900, 1000, 5000, 10000, 50000, 100000, 500000, 1000000, 5000000, or 10000000 cm³, or any volume that is within a range defined by any two of the aforementioned volumes, for example, 1 to 10000000 cm³, 500 to 1000000 cm³, 10000 to 100000 cm³, 1 to 100000 cm³, or 10000 to 10000000 cm³. In some embodiments, the collection channel (12) comprises a number of spheroids and liquid media appropriate for the volume of the collection channel to form a shaped gastrointestinal organoid with the size and dimensions appropriate for a human. In some embodiments, the collection channel (12) comprises a number of spheroids that is, is about, is at least, is at least about, is not more than, or is not more than about, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ spheroids, or any number of spheroids within a range defined by any two of the aforementioned numbers, for example, 10² to 10²⁰, 10⁵ to 10¹⁵, 10⁸ to 10¹², 10² to 10¹⁰, or 10¹⁰ to 10²⁰ spheroids. In some embodiments, the shaped gastrointestinal organoid is formed from a number of spheroids appropriate to form a shaped gastrointestinal organoid with the size and dimensions appropriate for a human. In some embodiments, the shaped gastrointestinal organoid is formed from a number of spheroids that is, is about, is at least, is at least about, is not more than, or is not more than about, 10², 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, or 10²⁰ spheroids, or any number of spheroids within a range defined by any two of the aforementioned numbers, for example, 10² to 10²⁰, 10⁵ to 10¹⁵, 10⁸ to 10¹², 10² to 10¹⁰, or 10¹⁰ to 10²⁰ spheroids. In some embodiments, the shaped gastrointestinal organoid that is appropriately configured for a human is a shaped esophageal organoid, shaped gastric organoid, shaped fundic gastric organoid, shaped antral gastric organoid, shaped small intestinal (intestinal) organoid, or shaped large intestinal (colonic) organoid. In some embodiments, the shaped gastrointestinal organoid that is appropriately configured for a human is a shaped intestinal organoid. In some embodiments, the shaped gastrointestinal organoid that is appropriately configured for a human is a shaped HIO.

As disclosed herein in some embodiments, by allowing spheroids to grow in vitro under certain conditions, shaped gastrointestinal organoids derived from PSCs are obtained. In some embodiments, the resulting shaped gastrointestinal organoids serve as a clinically beneficial tissue that can be used to study or treat a variety of different disease states, including but not limited to short gut, intestinal failure, necrotizing enterocolitis (NEC), injury, ulcers, Celiac disease, Crohn's disease, pathogenic infection, cancer, intestinal obstructions, or irritable bowel syndrome, or any combination thereof. In some embodiments, the resulting shaped gastrointestinal organoids are used to study esophageal, gastric, intestinal, or colonic function, including but not limited to drug screening, neurological function, microbiome interaction, or transplant, or any combination thereof. In some embodiments, the resulting shaped gastrointestinal organoids are used to study intestinal function. In some embodiments, the shaped gastrointestinal organoids comprise a functional lumen. In some embodiments, the shaped gastrointestinal organoids have the ability to further differentiate upon transplantation. In some embodiments, the shaped gastrointestinal organoids growth to the fetal stage in vitro and, upon transplantation, further differentiate. In some embodiments, the shaped gastrointestinal organoid is an elongated gastrointestinal organoid. In some embodiments, the shaped gastrointestinal organoid is an elongated intestinal organoid. In some embodiments, the shaped gastrointestinal organoid is an elongated HIO. In some embodiments, the shaped gastrointestinal organoid is prepared according to any one of the methods described herein using any one of the formation trays described herein.

Disclosed herein are methods of producing a shaped gastrointestinal organoid. In some embodiments, the shaped gastrointestinal organoid comprises a lumen. In some embodiments, the shaped gastrointestinal organoid is an elongated gastrointestinal organoid as described herein. In some embodiments, the methods comprise placing a plurality of spheroids into a collection channel comprising a predetermined shape and culturing the plurality of spheroids into the collection channel to differentiate the plurality of spheroids into the shaped gastrointestinal organoid having the predetermined shape. In some embodiments, the shaped gastrointestinal organoid comprises a mesenchyme and lumen. In some embodiments, the mesenchyme is a condensed mesenchyme. In some embodiments, the shaped gastrointestinal organoid undergoes spontaneous innervation. In some embodiments, the plurality of spheroids comprises a number of spheroids that is, is about, is at least, is at least about, is not more than, or is not more than about, 2500, 3000, 3500, 4000, 4500, or 5000, spheroids, or any number of spheroids within a range defined by any two of the aforementioned numbers, for example, 2500 to 5000 spheroids, 3000 to 4000 spheroids, 2500 to 4000 spheroids, or 3000 to 5000 spheroids. In some embodiments, the predetermined shape comprises a length. In some embodiments, the length is, is about, is at least, is at least about, is not more than, or is not more than about, 10, 11, 12, 13, 14, 15, 16, 17, 18, 19, 20, 21, 22, 23, 24, or 25 millimeters, or any length within a range defined by any two of the aforementioned lengths, for example, 10 to 25 mm, 15 to 20 mm, 10 to 20 mm, or 15 to 25 mm. In some embodiments, the length is an elongate length. In some embodiments, the predetermined shape comprises a diameter. In some embodiments, the diameter is, is about, is at least, is at least about, is not more than, or is not more than about 300 μm, 400 μm, 500 μm, 600 μm, 700 μm, 800 μm, 900 μm, or 1000 μm, or any diameter within a range defined by any two of the aforementioned diameters, for example, 300 μm to 1000 μm, 500 μm to 800 μm, 300 μm to 600 μm, or 500 μm to 1000 μm. In some embodiments, the ratio of the length to the diameter is, is about, is at least, is at least about, is not more than, or is not more than about 10, 20, 30, 40, 50, 60, 70, 80, 90, or 100, or any ratio between a range defined by any two of the aforementioned ratios, for example, 10 to 100, 30 to 80, 40 to 60, 10 to 50, or 50 to 100. In some embodiments, the volume of the gastrointestinal organoid is, is about, is at least, is at least about, is not more than, or is not more than about 0.1 mm³, 0.5 mm³, 1 mm³, 2 mm³, 3 mm³, 4 mm³, 5 mm³, 6 mm³, 7 mm³, 8 mm³, 9 mm³, 10 mm³, 11 mm³, 12 mm³, 13 mm³, 14 mm³, 15 mm³, 16 mm³, 17 mm³, 18 mm³, 19 mm³, 20 mm³, 21 mm³, 22 mm³, 23 mm³, 24 mm³, or 25 mm³, or any volume within a range defined by any two of the aforementioned volumes, for example, 0.1 mm³ to 25 mm³, 10 mm³ to 25 mm³, or 10 mm³ to 20 mm³. In some embodiments, the elongated gastrointestinal spheroid is comprised by or formed from spheroids that are gathered at a density that is, is about, is at least, is at least about, is not more than, or is not more than about, 100, 200, 300, 400, 500, 600, 700, 800, 900, 1000, 1100, 1200, 1300, 1400, 1500, 1600, 1700, 1800, 1900, or 2000 spheroids per mm³, or any density within a range defined by any two of the aforementioned densities, for example 100 to 2000 spheroids per mm³, 500 to 1500 spheroids per mm³, 100 to 1000 spheroids per mm³, or 1000 to 2000 spheroids per mm³. In some embodiments, the collection channel has a non-spherical shape and the shaped gastrointestinal organoid is a non-spherical gastrointestinal organoid. In some embodiments, the collection channel has an elongated shape and the shaped gastrointestinal organoid is an elongated gastrointestinal organoid. In some embodiments, the lumen is not continuous throughout the length of the shaped gastrointestinal organoid. In some embodiments, the shaped gastrointestinal organoid is a shaped human gastrointestinal organoid. In some embodiments, the shaped gastrointestinal organoid is derived from induced pluripotent stem cells reprogrammed from PBMC cells, a biopsy tissue sample, or Sendai virus-transduced somatic cells.

In some embodiments, the plurality of spheroids are cultured in the collection channel for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days. In some embodiments, the plurality of spheroids is cultured in a growth medium. In some embodiments, the growth medium is Advanced DMEM-F12. In some embodiments, the growth medium is Minigut media. In some embodiments, the growth medium is supplemented with EGF. In some embodiments, the growth medium is not supplemented with CHIR99021 or FGF4, or both. In some embodiments, the plurality of spheroids comprises a mesenchyme. In some embodiments, the plurality of spheroids fuse at the mesenchyme of the plurality of spheroids.

In some embodiments, the methods described herein further comprise inducing a mechanical strain on the shaped gastrointestinal organoid. In some embodiments, the mechanical strain promotes the spontaneous innervation of the shaped gastrointestinal organoid. In some embodiments, the mechanical strain decreases maturation time of the shaped gastrointestinal organoid. In some embodiments, the mechanical strain is a uniaxial tensile strain.

In some embodiments, the shaped gastrointestinal organoid further comprises enteric neuronal cells or enteric neuronal progenitor cells, or both. In some embodiments, the shaped gastrointestinal organoid comprises one or more myenteric plexuses. In some embodiments, the one or more myenteric plexuses comprise cells that express the neuronal marker PGP9.5. In some embodiments, the shaped gastrointestinal organoid has neuronal activity. In some embodiments, the shaped gastrointestinal organoid comprises a polarized, columnar epithelium surrounded by mesenchyme. In some embodiments, the mesenchyme comprises a smooth muscle-like layer. In some embodiments, the shaped gastrointestinal organoid comprises an epithelium patterned into crypt-like proliferative zones or villus-like structures, or both. In some embodiments, the shaped gastrointestinal organoid comprises laminated longitudinal and circular muscle. In some embodiments, the shaped gastrointestinal organoid comprises markers of smooth muscle or intestinal sub-epithelial myofibroblasts, or both. In some embodiments, the shaped gastrointestinal organoid comprises one or more of enterocytes, enteroendocrine cells, goblet cells, Paneth cells, or any combination thereof. In some embodiments, the shaped gastrointestinal organoid comprises cells that express one or more of villin, Muc2, DEFA5, CHGA, or OLFM4, or any combination thereof. In some embodiments, the shaped gastrointestinal organoid is vascularized in vitro. In some embodiments, the shaped gastrointestinal organoid is vascularized upon engraftment into an individual.

Described herein are embodiments of formation trays. In some embodiments, the formation tray is used for culturing one or more gastrointestinal organoids. In some embodiments, the formation tray is used for culturing one or more shaped gastrointestinal organoids. In some embodiments, the formation tray is used for culturing one or more elongated gastrointestinal organoids. In some embodiments, the formation tray comprises one or more collection channels configured to receive one or more plurality of spheroids therein. In some embodiments, the one or more collection channels have an elongated shape. In some embodiments, the one or more collection channels have a non-spherical shape. In some embodiments, the one or more collection channels are configured to gather the one or more plurality of spheroids together such that the one or more plurality of spheroids define a predetermined shape. In some embodiments, the one or more spheroids differentiate into the one or more gastrointestinal organoids having the predetermined shape. In some embodiments, the one or more spheroids differentiate into the one or more elongated gastrointestinal organoids having the predetermined shape. In some embodiments, the one or more collection channels are made of a biocompatible material configured to inhibit the one or more plurality of spheroids from attaching thereto. In some embodiments, the one or more collection channels comprise one or more plurality of spheroids positioned therein. In some embodiments, the one or more collection channels comprise a cell culture media or extracellular matrix, or both, therein. In some embodiments, the one or more collection channels further comprise the one or more gastrointestinal organoids positioned therein. In some embodiments, the one or more gastrointestinal organoids is one or more shaped gastrointestinal organoid produced by any one of the methods described herein.

Described herein are embodiments of kits. In some embodiments, the kit is used for culturing a gastrointestinal organoid. In some embodiments, the kit comprises a formation tray comprising one or more collection channels. In some embodiments, the formation tray is any one of the formation trays described herein. In some embodiments, the kit comprises a plurality of spheroids configured to be received within the one or more collection channels of the formation tray. In some embodiments, the kit comprises a cell culture media configured to be received within the one or more collection channels of the formation tray.

### Transplantation and Methods of Treatment

In some embodiments, after the predetermined period of formation time and generation of an shaped gastrointestinal organoid as described herein, the shaped gastrointestinal organoid is transplanted into a host organism, for example, as a treatment or an experimental model, as described herein. In some embodiments, the shaped gastrointestinal organoid is an elongated gastrointestinal organoid. In one embodiment, after the predetermined period of formation time and generation of an shaped gastrointestinal organoid (46), the shaped gastrointestinal organoid (46) is transplanted into a host organism (44) as shown in FIG. 5. In some embodiments, the transplant is performed after culturing the organoid for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 days, or any number of days of culture within a range defined by any two of the aforementioned days, for example, 1 to 50 days, 10 to 40 days, 20 to 30 days, 1 to 30 days, or 20 to 50 days. In some embodiments, the transplant is performed after culturing the organoid for a number of days that is, is about, is at least, is at least about, is not more than, or is not more than about, 11, 12, 13, 14, 15, 16, or 17 days. In some embodiments, the shaped gastrointestinal organoid is mature enough for transplantation and/or study a number of days before gastrointestinal organoids prepared by other methods known in the art are at the same or similar mature state, wherein the number of days is, is about, is at least, is at least about, is not more than, or is not more than about, 1, 2, 3, 4, 5, 6, 7, 8, 9, 10, 11, 12, 13, 14, 15, 16, 17, 18, 19, or 20 days, or any number of days within a range defined by any two of the aforementioned number of days, for example, 1 to 20 days, 5 to 15 days, 10 to 15 days, 1 to 15 days, or 10 to 20 days. In some embodiments, the host organism is a mammal. In some embodiments, the host organism is an immunodeficient mammal. In some embodiments, the host organism is an immunodeficient mouse. In some embodiments, the host organism is a monkey, dog, hamster, or rat. In some embodiments, the host organism is an immunocompromised monkey, dog, hamster, or rat. In some embodiments, the host organism is a human. In some embodiments, the host organism is an immunodeficient human. In some embodiments, the host organism is an immunocompetent human. In some embodiments, the host organism is an immunocompetent human treated with immunosuppressants. In some embodiments, the host organism is an immunocompetent human and the shaped gastrointestinal organoid is autologous to the host organism. In some embodiments, the host organism is an immunocompetent human and the shaped gastrointestinal organoid is allogeneic to the host organism. In some embodiments, the host organism is a mammal that is in need of a gastrointestinal organ transplant. In some embodiments, the host organism is a human that is in need of a gastrointestinal organ transplant. In some embodiments, the gastrointestinal organoid is not intended to be unnecessarily limited to the shaped gastrointestinal organoid shown as (46) or described herein.

In some embodiments, the gastrointestinal organoid is a generally spherical gastrointestinal organoid, a shaped gastrointestinal organoid, or an elongated gastrointestinal organoid as described herein. In some embodiments, the gastrointestinal organoid is implanted adjacent to the bowel of the animal. In some embodiments, the gastrointestinal organoid is implanted on top of the mesenteric vasculature of the animal. In some embodiments, the gastrointestinal organoid is secured with an adhesive. In some embodiments, the adhesive is a cyanoacrylate glue. In some embodiments, the gastrointestinal organoid is connected to the gastrointestinal tract of an animal through an organoid-to-intestine anastomosis. In some embodiments, the anastomosis is a side-to-side anastomosis or an end-to-end anastomosis. In some embodiments, the gastrointestinal organoid grows in the animal for a number of days that is, is about, is at least, is at least about, is not more than, or is not more than about, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 days. In some embodiments, the gastrointestinal organoid grows larger than in vitro gastrointestinal organoids prepared at the same time. In some embodiments, the gastrointestinal organoid exhibits integration with the host organism tissue.

In some embodiments, the gastrointestinal organoid comprises gastrointestinal cell lineages. In some embodiments, the gastrointestinal organoid comprises one or more of mesenchyme, mucus cells, parietal cells, chief cells, gastrin cells, alveolar cells, enterocytes, enteroendocrine cells, goblet cells, microfold cells, cup cells, tuft cells, or Paneth cells, or any combination thereof. In some embodiments, the gastrointestinal organoid comprises cells that express one or more (e.g. 1, 3, 5) of VILLIN, MUC2, DEFA5, CHGA, or OLFM4, or any combination thereof. In some embodiments, the gastrointestinal organoid develops gastrointestinal cell lineages spontaneously.

In some embodiments, the intestinal organoid comprises gastrointestinal cell lineages. In some embodiments, the gastrointestinal organoid comprises one or more of mesenchyme, enterocytes, enteroendocrine cells, goblet cells, or Paneth cells, or any combination thereof. In some embodiments, the intestinal organoid comprises cells that express one or more (e.g. 1, 3, 5) of VILLIN, MUC2, DEFA5, CHGA, or OLFM4, or any combination thereof. In some embodiments, the intestinal organoid develops intestinal cell lineages spontaneously.

In some embodiments, the gastrointestinal organoid comprises neuronal structures. In some embodiments, the gastrointestinal organoid comprises cells that express neuronal markers. In some embodiments, the gastrointestinal organoid comprises cells that express PGP9.5. In some embodiments, the gastrointestinal organoid comprising neuronal structures or cells expressing neuronal markers was not combined with any neuronal lineage cells, such as neural crest cells during its formation. In some embodiments, the gastrointestinal organoid develops neuronal structures spontaneously. In some embodiments, the gastrointestinal organoid becomes innervated spontaneously. In some embodiments, the gastrointestinal organoid becomes innervated without experiencing mechanical strain. In some embodiments, the gastrointestinal organoid comprises one or more myenteric plexuses. In some embodiments, the gastrointestinal organoid develops one or more myenteric plexuses spontaneously. In some embodiments, the myenteric plexus size of the gastrointestinal organoid is larger than the myenteric plexus size of gastrointestinal organoids combined with neural crest cells according to previous methods (e.g. the methods seen in PCT Publication WO 2016/061464). In some embodiments, the gastrointestinal organoid comprises one or more myenteric plexuses at a percentage of total cell density that is, is about, is at least, is at least about, is not more than, or is not more than about, 1%, 2%, 3%, 4%, 5%, 6%, 7%, 8%, 9%, 10%, 11%, 12%, 13%, 14%, 15%, 16%, 17%, 18%, 19%, or 20% of the total cell density, or any percentage within a range defined by any two of the aforementioned percentages, for example, 1% to 20%, 5% to 15%, 8% to 12%, 1% to 15%, or 10% to 20%.

In some embodiments, the gastrointestinal organoid comprises vascular or endothelial structures. In some embodiments, the gastrointestinal organoid comprises cells that express vascular or endothelial markers. In some embodiments, the gastrointestinal organ was not combined with any endothelial lineage cells. In some embodiments, the gastrointestinal organoid develops vascular or endothelial structures spontaneous. In some embodiments, the gastrointestinal organoid becomes vascularized spontaneously. In some embodiments, the vascular or endothelial structure is originated from the host organism.

In some embodiments, the gastrointestinal organoid comprises a lumen. In some embodiments, the gastrointestinal organoid comprises a lumen that occupies a percentage of the total volume of the gastrointestinal organoid. In some embodiments, the lumen occupies a percentage of the total volume of the gastrointestinal organoid that is, is about, is at least, is at least about, is not more than, or is not more than about, 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 38%, 39%, or 40% of the total volume of the gastrointestinal organoid, or any percentage within a range defined by any two of the aforementioned percentages, for example, 1% to 40%, 10% to 30%, 15% to 20%, 1% to 20%, or 10% to 40%.

In some embodiments, the gastrointestinal organoid exhibits upregulation of genes relative to an organoid produced by conventional methods. In some embodiments, the gastrointestinal organoid exhibits upregulation of a number of genes that is, is about, is at least, is at least about, is not more than, or is not more than about, 100, 150, 200, 250, 300, 350, 400, 450, 500, 550, 600, 650, 700, 750, or 800 genes relative to an organoid produced by conventional methods, or any number of genes within a range defined by any two of the aforementioned number of genes, for example, 100 to 800 genes, 200 to 600 genes, 300 to 500 genes, 100 to 400 genes, or 400 to 800 genes. In some embodiments, the gastrointestinal organoid exhibits downregulation of a number of genes that is, is about, is at least, is at least about, is not more than, or is not more than about, 100, 150, 200, 250, 300, 350, 400, 450, 500, 550, 600, 650, 700, 750, 800, 850, 900, 950, 1000, 1100, 1200, 1300, 1400, 1500, 1600, 1700, 1800, 1900, or 2000 genes relative to an organoid produced by conventional methods, or any number of genes within a range defined by any two of the aforementioned number of genes, for example, 100 to 2000 genes, 400 to 1500 genes, 700 to 1000 genes, 100 to 1000 genes, or 1000 to 2000 genes. In some embodiments, the genes that are upregulated are involved in one or more (e.g., at least 1, 3, 5, 10) of neuron differentiation, neurogenesis, generation of neurons, neuron projection development, regulation of multicellular organismal development, neuron development, neuron projection morphogenesis, cell adhesion, axon development, or biological adhesion, or any combination thereof. In some embodiments, the genes that are upregulated are involved in one or more (e.g., at least 1, 3, 5, 10) of pattern specification processes, regionalization, anterior/posterior pattern specification, anatomical structure formation involved in morphogenesis, animal organ morphogenesis, embryo development, tube morphogenesis, epithelium development, epithelial tube morphogenesis, embryonic morphogenesis, circulatory system development, positive regulation of multicellular organismal processes, regulation of multicellular organismal development, tube development, vasculature development, regulation of cell differentiation, blood vessel development, positive regulation of developmental processes, digestive tract development, extracellular matrix organization, extracellular structure organization, inflammatory response, biological adhesion, cell adhesion, response to wounding, regulation of cell proliferation, defense response, regulation of cell migration, regulation of locomotion, neuron differentiation, generation of neurons, neurogenesis, neuron projection development, neuron development, regulation of multicellular organismal development, cell adhesion, biological adhesion, neuron projection morphogenesis, or cell projection organization, or any combination thereof.

Described herein are methods of treating an individual having compromised gastrointestinal function, or ameliorating or inhibiting a detrimental gastrointestinal disorder in an individual in need thereof. In some embodiments, the methods comprise transplanting or engrafting a gastrointestinal organoid into the individual. In some embodiments, the gastrointestinal organoid is a gastrointestinal organoid of any one of the methods described herein. In some embodiments, the gastrointestinal organoid is an esophageal organoid, gastric organoid, fundic gastric organoid, antral gastric organoid, small intestinal (intestinal) organoid, or large intestinal (colonic) organoid. In some embodiments, the gastrointestinal organoid is an intestinal organoid. In some embodiments, the gastrointestinal organoid is an HIO. In some embodiments, the gastrointestinal organoid is a shaped gastrointestinal organoid of any one of the methods described herein. In some embodiments, the gastrointestinal organoid is a shaped or elongated gastrointestinal organoid of any one of the methods described herein. In some embodiments, the gastrointestinal organoid is autologous or allogeneic to the individual. In some embodiments, the gastrointestinal organoid is prepared from induced pluripotent cells obtained or derived from the individual. In some embodiments, the individual is in need of a gastrointestinal transplant. In some embodiments, the gastrointestinal organoid is transplanted or engrafted as a whole gastrointestinal organoid. In some embodiments, the transplant site is a gastrointestinal tissue.

### EXAMPLES

Some aspects of the embodiments discussed above are disclosed in further detail in the following examples, which are not in any way intended to limit the scope of the present disclosure. Those in the art will appreciate that many other embodiments also fall within the scope of the invention, as it is described herein above and in the claims.

### Example 1. Generation of iPSCs from Human Somatic Tissue (Biopsy or Blood)

Human somatic cells were collected and utilized for iPSC generation. Either a peripheral blood mononuclear cell (PBMC) fraction from fresh whole blood by Ficoll centrifugation or thawed cryopreserved PBMCs were starting material. PBMCs were plated at 1-5×10⁶ cells in 2 mL of Erythroid Expansion Media (EEM) into a single well of a 6-well dish and incubated for 24 hours at 37° C., 5% CO₂. On day 2, the 2 mL suspension was transferred to a new 6-well dish to select for the non-adherent cells. The non-adherent cells were incubated for 5 days at 37° C., 5% CO₂. Over the next 5 days, 1 mL of fresh EEM was added to each cell-containing well every other day. On day 7, 2 mL of 0.1% gelatin was added to a new 6-well plate per donor and placed in a 37° C., 5% CO₂ incubator overnight. On day 8, about 187,500 irradiated mouse embryonic fibroblasts (MEFs) were placed onto the gelatin-coated 6-well plates (1 well per PBMC donor).

On day 9, PBMCs were transduced with Sendai virus. For each sample to be transduced, 3×10⁵ cells were transferred to a 14 mL round bottom tube in a volume no greater than 500 μL of EEM. A Sendai virus master mix comprising human Klf4, Oct3/4, Sox2 (KOS), human L-Myc (hL-Myc), and human Klf4 (hKlf4) transgenes (CTS CytoTune 2.1, Invitrogen) were prepared at a component ratio of (MOI×number of cells)/(titer of virus×10⁻³ (mL/μL)), where MOI of KOS is 2.5, MOI of hL-Myc is 2.5, and MOI of hKlf4 is 1.5. MOI [multiplicity of infection] refers to the cell infection units (CIU) per cell, and the titer of virus varies between preparations of the virus. Master mix is warmed to 37° C. 1 mL of Sendai virus master mix is added to each sample in the round bottom tubes. Samples are centrifuged at 1000×g for 30 minutes at room temperature. After centrifugation, 1 mL of warm EEM is added to each tube, the cells are gently resuspended, and the entire volume is plated into a well of a 12-well plate. The plate containing the samples is placed in a 37° C., 5% CO₂ incubator.

On day 10, the cells and medium are collected from the 12-well plate and transferred to a 15 mL conical tube. The wells are rinsed with 1 mL of fresh EEM to ensure that all of the cells have been collected. The tubes are centrifuged at 200×g for 5 minutes at room temperature to remove Sendai virus from the cells. After centrifugation, the supernatant is discarded into 15% bleach disinfectant to inactivate the virus. The cell pellet is resuspended in 0.5 mL of EEM and plated onto a well of a 24-well plate. The cells are incubated for 48 hours at 37° C., 5% CO₂. A gelatin coated 6-well dish is prepared for each sample that was transduced according to the process above.

On day 11, the gelatin is aspirated from the gelatin coated plates and immediately, about 187,500 irradiated MEFs are plated in MEF media on to the gelatin coated plates. The MEFs are incubated for 24 hours at 37° C., 5% CO₂.

On day 12, MEF media is removed and the MEF plate is rinsed two times with 2 mL of PBS for each well. 2.5 mL of StemPro 34 SFM media is added to each MEF well. A live cell count is performed on transduced samples to determine the total number of PBMCs. The PBMCs are re-plated onto the MEF-coated wells at 4 concentrations (cells/well): 5×10³, 1×10⁵, 2.5×10⁵, and 5×10⁵. The cells are incubated for 24 hours at 37° C., 5% CO₂.

On day 13 and 15, a 50% media change is performed by removing ~50% of the media from the wells and adding an equal volume of fresh StemPro 34 SFM media.

On day 16, a 50% media change is performed by removing ~50% of the media from the wells and adding an equal volume of StemPro hESC media+bFGF (2 μg/mL).

On day 17-40, a full media change is performed daily with fresh hESC media+bFGF (2 μg/mL) and the cells are monitored for colony formation (generally around 21-28 days). Once the cells reach the desired state, they can be frozen in 1 mL of a cryopreservation media in a 1.5 mL cryovial at approximately 1-2 million cells per vial for future use.

### Example 2. Generation of Un-Shaped Human Intestinal Organoids (HIOs)

Human PSCs, which can be either hESCs or hiPSCs, are cultured in feeder-free conditions in 6-well Nunclon Delta surface-treated tissue culture dishes (Nunc) coated with hESC-qualified Matrigel (Corning) and maintained in mTeSR1 medium (StemCell Technologies). hPSCs are first passaged with either Dispase (Thermo Fisher) for “clump passaging” or Accutase (Thermo Fisher) for “single cell passaging” and are then re-plated at “high” or “low” confluence in a hESC-qualified Matrigel-coated Nunclon 24-well plate with mTeSR1 medium. mTeSR1 medium for hPSCs undergoing single cell passaging is supplemented with 10 μM Y-27632 dihydrochloride (a Rho-associated, coiled-coil containing protein kinase (ROCK) inhibitor, Tocris) for the first day only. hPSCs passaged at low confluence receive a second day of mTeSR1 medium to allow the monolayer to reach 80-95% confluence, whereas hPSCs passage at high confluence are expected to already be at 80-95% confluence after the first day.

Cells are differentiated into definitive endoderm by treating with 100 ng/mL of Activin A (Cell Guidance Systems) in RPMI 1640 medium (Invitrogen) for three days. The RPMI 1640 medium is supplemented with 1×NEAA (Invitrogen) and increasing concentrations of defined FBS (dFBS, Hyclone) at 0%, 0.2%, and 2.0% on the first, second, and third days of Activin A treatment, respectively. Additionally, low concentrations of BMP4 (1-15 ng/mL of BMP4 (R&D Systems) may or may not be supplemented on the first day of Activin A treatment. Following this, the DE monolayer is then treated with mid-hindgut spheroid induction medium for four days. The mid-hindgut spheroid induction medium comprises 3 μM CHIR99021 (a glycogen synthase kinase 3 (GSK3) inhibitor, Stemgent) and 500 ng/mL of FGF4 (R&D Systems) in RPMI 1640 supplemented with 1×NEAA and 2.0% dFBS.

After four days of mid-hindgut spheroid induction, free-floating spheroids are collected and embedded in a 3D basement membrane Matrigel “dome/bubble” and then subsequently maintained in basal gut medium. Basal gut medium comprises Advanced DMEM-F12 (Invitrogen), 1×N2 supplement (Invitrogen), 1×B27 supplement without vitamin A (Thermo Fisher), 15 mM HEPES (Life Technologies), 2 mM L-glutamine (Life Technologies), and 100 units/mL (1×) penicillin-streptomycin (Life Technologies) supplemented with 100 ng/mL epidermal growth factor (EGF, R&D Systems). Medium is changed every 3-4 days or whenever the medium turns yellow due to pH, whichever occurs first, for approximately two weeks. HIOs are then re-plated in fresh Matrigel with fewer organoids per Matrigel dome to allow for continued expansion. The same basal gut medium treatment schedule is maintained typically for another two weeks with prolonged culturing being possible.

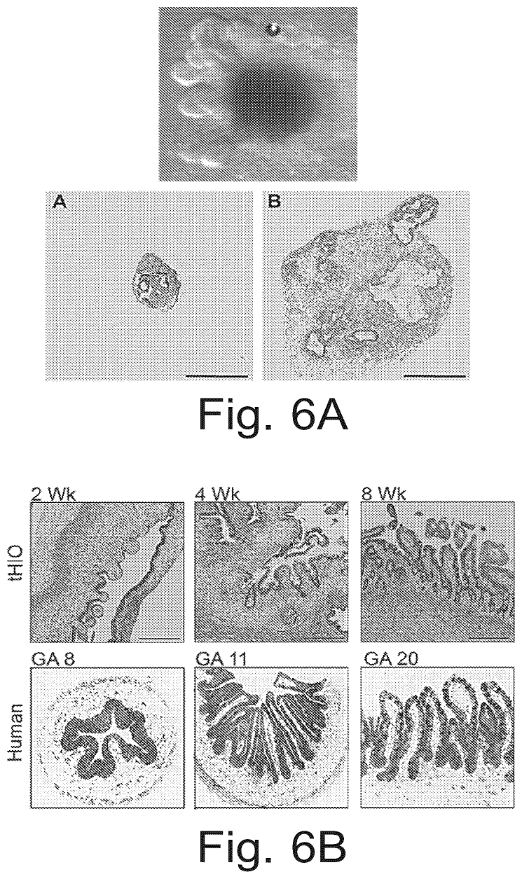

The resulting HIOs are three-dimensional structures (FIG. 6A) comprising a polarized, columnar epithelium surrounded by mesenchyme that includes a smooth muscle-like layer. The epithelium is patterned into crypt-like proliferative zones and villus-like structures and the mesenchyme into laminated longitudinal and circular muscle as well as the lamina propria with all of the major functional cell types of the intestine. Additionally, organoids cultured with the method herein contain a stratified mesenchyme and express markers of smooth muscle and intestinal sub-epithelial myofibroblast cells vital to the ability of these tissues to engraft in the intestine, and also resemble fetal intestine morphology (FIG. 6B). HIO mesenchyme differentiation precedes epithelial differentiation, indicating that they create and understand their own niche.

### Example 3. Generation of Shaped Elongated HIOs

An in vitro confinement protocol using the collection channel embodiments described herein generate continuous cylindrical organoid structures suitable for transplantation, such as into immunocompromised animal models. It is observed that the required amount of time in vitro prior to successful engraftment is reduced by approximately 14 days when compared to the HIO generation protocol of Example 2.

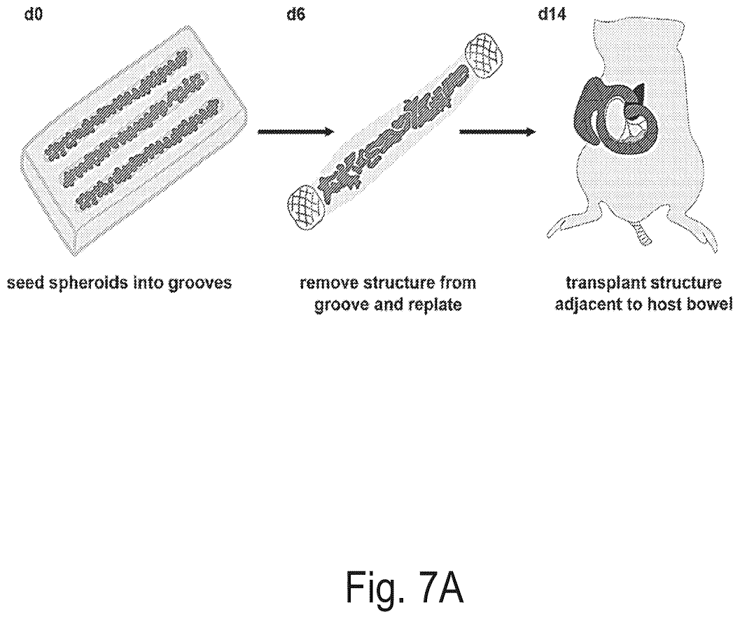

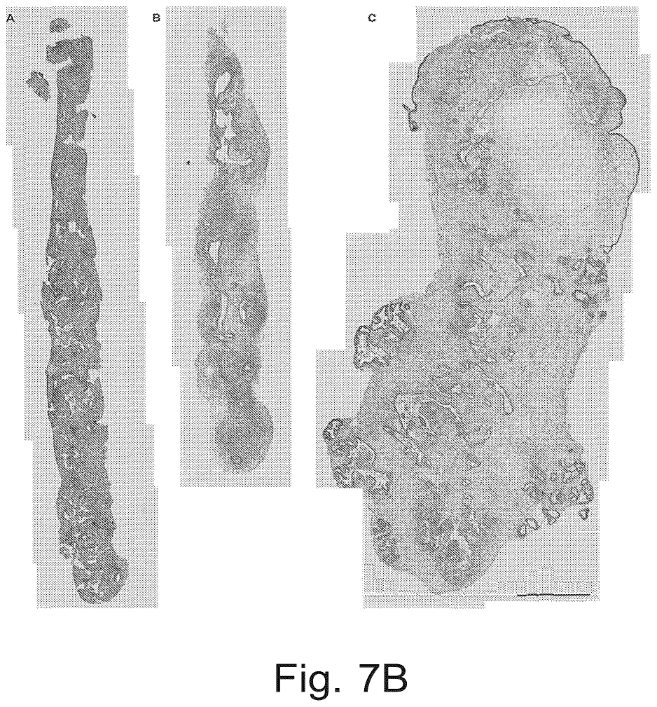

FIG. 7A depicts the methodology of forming the cylindrical intestinal organoid structures. hPSCs were cultured, induced to definitive endoderm, and differentiated into intestinal spheroids as described herein. Upon collecting the spheroids, they were filtered through a 70 μm pore size, retaining the spheroids that are larger than 70 μm and discarding those that are smaller. This size cutoff appears to have better capacity to form HIOs, but may differ for other tissue types. The retained spheroids were resuspended in 2 mL of Minigut media, and a 50 μL sample was taken to quantify the number of spheroids by microscopy. Based on the quantification, the total number of spheroids was estimated. Approximately 3000-4000 spheroids were seeded per groove of the collection channel in 50% Matrigel diluted with Minigut media. The number of spheroids used will depend on the geometry of the groove; the spheroids should be densely packed in the groove. The parameters disclosed herein are for a groove with a hemispheric cross section with a diameter of 0.5 mm and length of 15 mm.

The collection channel containing the spheroids was incubated at 37° C. for 30-45 minutes. To each collection channel, 5 mL of Minigut media supplemented with 100 ng/mL EGF was added. The media was changed for fresh media on day 4 of culture. On day six of culture, Dumont #4 forceps were used to carefully remove the organoid structure from the groove. The organoid structure was placed into the well of a Tissue Train Culture Plate, aligning the structure between the nylon mesh tabs. 200-400 μL of growth factor reduced (GFR) Matrigel (Corning) was added to the plate, covering both nylon mesh tabs and organoid structure in between. The plate was incubated at 37° C. for 90 minutes. To each well, 6 mL of Minigut media supplemented with 100 ng/mL EGF was added. The media was changed for fresh media twice weekly until day 14.

FIG. 7B shows the progression of in vitro growth of the elongated intestinal organoid shaped in the collection channel groove (g-HIO) by immunohistochemistry. Hematoxylin and eosin stained sections of day 6, day 14, and day 28 g-HIO structures.

Materials: mTeSR1 media (StemCell Technologies); Advanced DMEM-F12 (Invitrogen); RPMI 1640 (Invitrogen); hESC qualified Matrigel (Corning); Defined FBS (Hyclone); L-glutamine (100×) (Invitrogen); penicillin-streptomycin (100×) (Invitrogen); 50×B27 supplement (Invitrogen); HEPES buffer (Invitrogen); Dispase (Invitrogen); Activin A (R&D Systems); FGF4 (R&D Systems); CHIR99021 (R&D Systems); polydimethylsiloxane (PDMS) tissue culture collection channel scaffold with appropriately sized grooves; GFR Matrigel, phenol red-free (Corning); Minigut media: Advanced DMEM-F12 medium supplemented with 2 mM glutamine, 10 mM HEPES, 100 U/mL penicillin, 100 μg/mL streptomycin, 1×N2 supplement, 1×B27 supplement; human recombinant EGF (R&D Systems); Dumont #4 forceps (Fine Science Tools); Tissue Train Culture Plates with nylon mesh anchors (FlexCell International).

It is envisioned that alternative collection channel scaffolds can be constructed using machining processes or a 3D printer with a wide range of materials, such as metal, glass, plastic (e.g. acrylonitrile butadiene styrene (ABS), PLA, PP, PC, PS, PET, nylons, PE, polyurethanes, PVC, PVDC, PTFE, polyesters, PMMA, PEEK, PEI). In some embodiments, the scaffold is initially made and used to prepare a mold, so that a similarly shaped scaffold can be made out of a biologically inert or generally biologically inert material such as PDMS or other silicone.

### Example 4. Transplantation of HIOs

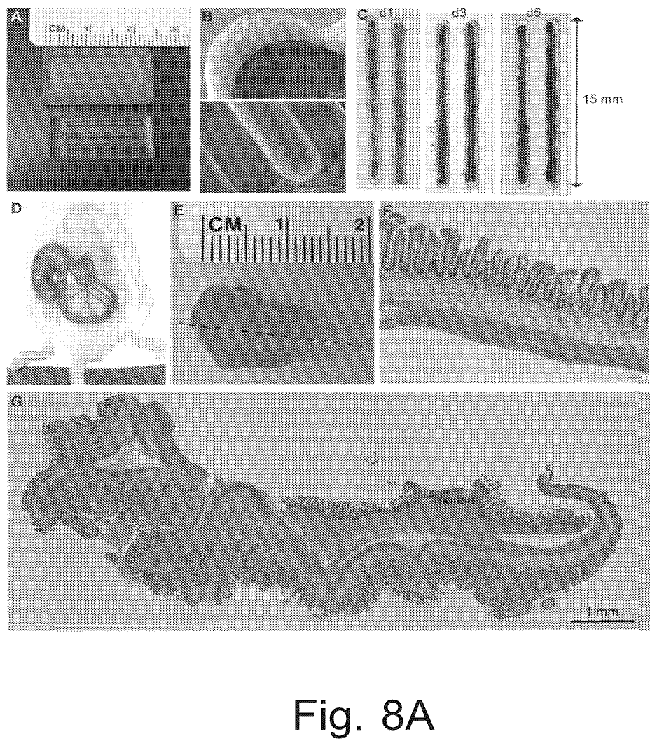

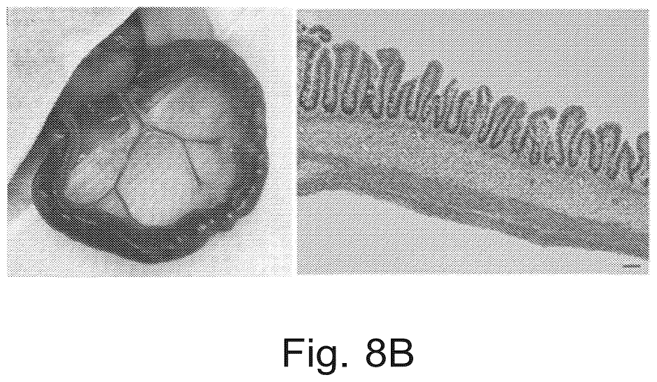

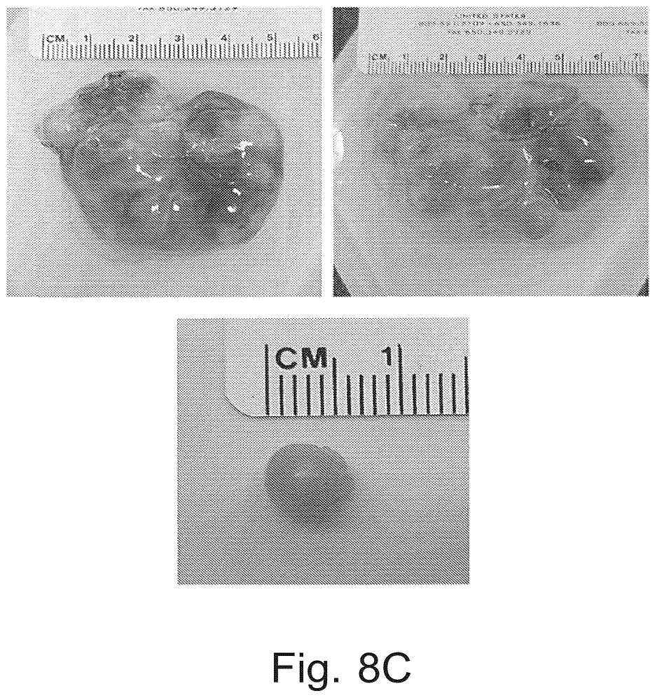

Immunocompromised mice were kept on antibiotic chow (275 ppm sulfamethoxazole and 1365 ppm trimethoprim). Food and water were provided ad libitum before and after surgery. Mice were anesthetized with 2% inhaled isoflurane and the abdominal wall was prepped in a sterile fashion with isopropyl alcohol and povidone-iodine. A 1-2 cm midline incision was made to gain access to the abdominal cavity. The cecum was identified and gently pulled out with the colon and small intestine following. The mesentery was splayed out with identification of the distal ileum and the ascending colon. At a location with bifurcating mesenteric vessels 1-2 arcades from the ileocecal junction, a single drop of octyl/butyl cyanoacrylate adhesive glue was place and the HIO was dropped onto the glue and allowed to dry in place for a minimum of 3 minutes. The organoid structure was positioned adjacent to the bowel overtop mesenteric vasculature. The intestines were then returned to the abdominal cavity and the mice were given an intraperitoneal flush of piperacillin/tazobactam (100 mg/kg). The skin was closed in a double layer and the mice were given a subcutaneous injection of Buprenex (0.05 mg/kg). Survival of mice was followed out to 10 weeks at which time the mice were humanely euthanized. The organoid grafts were excised and processed for histology. Successful engraftment of the organoid and integration of human PSC-derived tissue with the adjacent mouse host tissue is observed (FIGS. 8A-B). Whole shaped g-HIOs showed successful engraftment and vascularization when implanted into immunocompromised rats (FIG. 8C). Transplantation of unshaped HIOs prepared according to previous methods (e.g. in WO 2016/061464) did not engraft into immunocompromised rats.

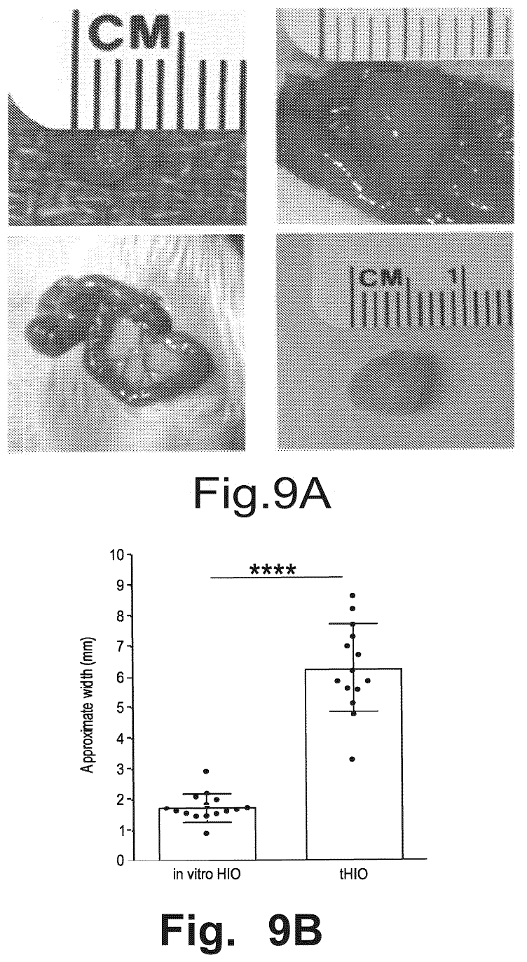

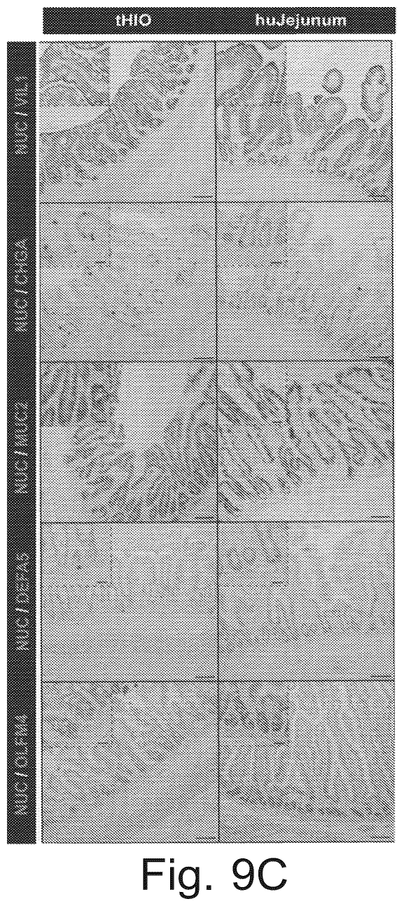

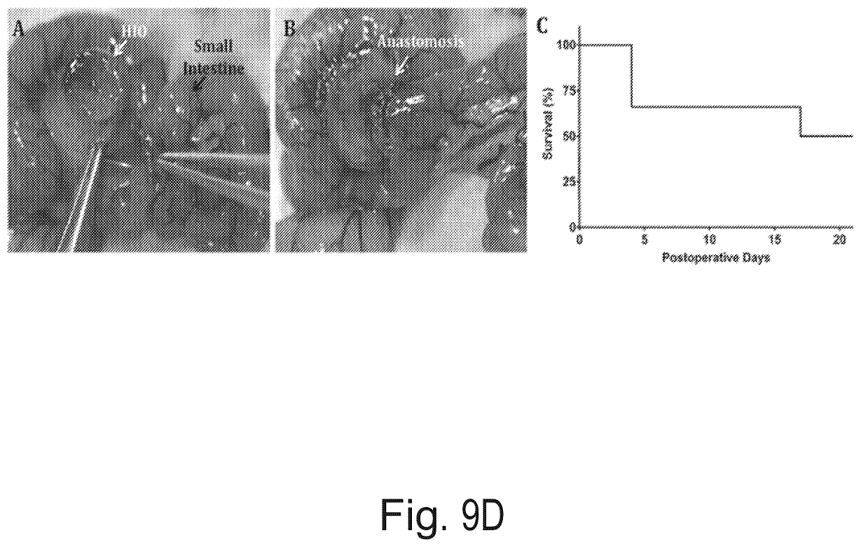

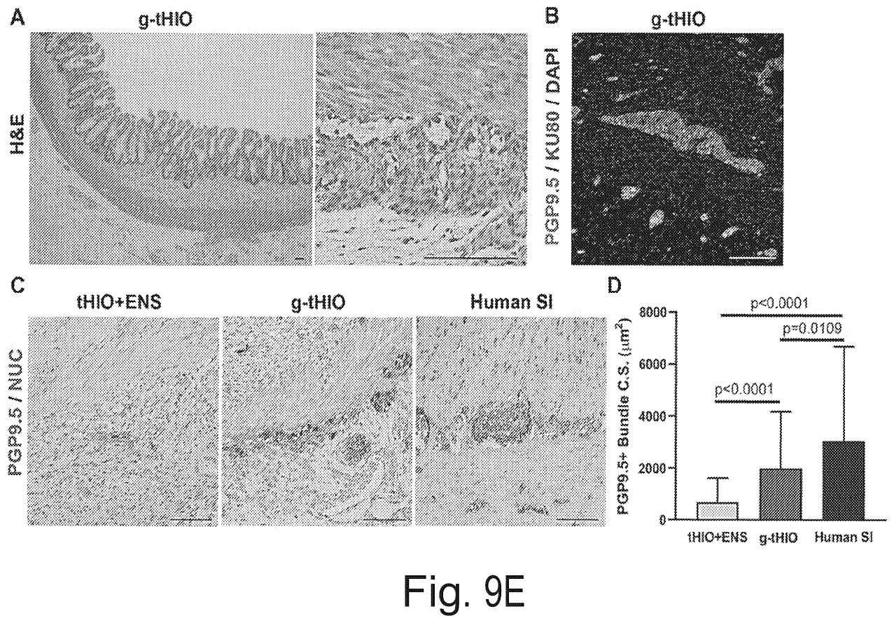

Percent engraftment and size of the HIO were measured. Overall survival rate was 85% (n=17/20) and 82% (n=14/17) had a successful HIO engraftment with host mesentery. The transplanted organoids (tHIO) were approximately 46 times larger than time matched in vitro HIOs (FIG. 9A-B). Histological analysis of this confirmed native appearing mesenchyme with subepithelial elements and muscular layers, as well as a continuous expansion of the epithelium with the presence of the major cell lineages including mesenchyme, enterocytes, enteroendocrine cells, goblet cells, and Paneth cells, similar to human intestine (FIG. 9C).

Optionally, mice underwent an organoid-to-intestine anastomosis. In a second surgery following the initial organoid transplant, the organoid and adjacent small bowel were identified and removed from the abdominal cavity. A side-to-side anastomosis was performed using 9-0 nylon in an interrupted fashion. Upon completion, the anastomosis was evaluated for gross leakage and the intestines were replaced into the abdominal cavity, taking care to avoid torsion. 50% of mice (n=6) survived to 21 days at the time of harvest (FIG. 9D).

After additional inspection, elongated intestinal organoids that were implanted into NSG mice after culturing in the collection channel (groove-tHIO, g-tHIO) spontaneously developed neuronal structures upon engraftment (FIG. 9E, using an anti-PGP9.5 antibody as a pan-neuronal marker). A robust network of myenteric plexuses, the major collection of neurons of the enteric nervous system, is observed throughout the transplanted g-tHIO. It is been previously shown that intestinal organoids can be innervated by mechanically aggregating mid-hindgut spheroids with PSC-derived neural crest cells (NCCs) (see WO 2016/061464). It is now demonstrated herein that intestinal organoids produced from PSCs and prepared according to the protocols provided herein develop enteric nervous system structures without the addition of separate NCCs. Furthermore, the plexus size of implanted g-tHIOs is consistently larger than those seen in the previous spheroid/NCC aggregate organoids.

Materials: Mice: female or male immunocompromised NSG (NOD-scid IL2Rgamma^{null}) mice were housed in microisolator systems in a barrier facility. The mice used were between 6 and 14 weeks of age. It is envisioned that other immunocompromised animal models, such as other immunocompromised mice models or immunocompromised monkey, dog, hamster, or rat models. Diet: A modified chow diet (Picolab Rodent Diet 20, LabDiet) is supplemented with 275 ppm sulfamethoxazole and 1365 ppm trimethoprim (LabDiet). A liquid diet is used for the side-to-side anastomosis surgery (Jevity 1 Cal). 0.3 mg/mL of 275 ppm sulfamethoxazole and 1365 ppm trimethoprim (Bactrim) are diluted in sterile water and given ad libitum after the side-to-side anastomosis. Antibacterial drugs: 100 mg/kg of piperacillin and tazobactam are diluted in sterile saline solution and used for any surgeries (ZOSYN, Pfizer). Surgical instruments (Fine Science Tools): suture tying forceps, ring forceps, dissecting scissors, Bishop-Harmon forceps, Halsey needle holder, sterilization tray. Isoflurane and anesthesia system. Sterile 7-0 nonabsorbable silk suture (PERMA-HAND), sterile 4-0 coated absorbable suture (VICRYL RAPIDE), sterile 9-0 nonabsorbable nylon suture with taper cut needle (ETHILON), octyl/butyl cyanoacrylate topical tissue adhesive (GLUture).

Immunohistochemistry: Transplanted HIOs were harvested and fixed overnight in 4% paraformaldehyde (PFA), then processed and embedded in paraffin. Slides of 5 m thick sections of tissue were made and deparaffinized, followed by heat-induced epitope retrieval and staining. Incubation for both primary and secondary antibodies took place at 4° C. overnight in 1% bovine serum albumin in phosphate buffered saline (PBS). The following primary antibodies and their respective dilutions were used: goat anti-villin (1:100), mouse anti-HuMuc2 (1:1250), mouse anti-DEFA5 (1:500), mouse anti-CHGA (1:500) and rabbit anti-OLFM4 (1:400). The following secondary antibodies were used: horse anti-goat biotin (1:1000), horse anti-mouse biotin (1:1000) and goat anti-rabbit biotin (1:1000). A peroxidase-based detection system was used followed by nuclear fast red (NUC) as counterstain (Vector Labs; Polysciences, Inc).

### Example 5. Transcriptomic Profiling of Shaped Intestinal Organoids

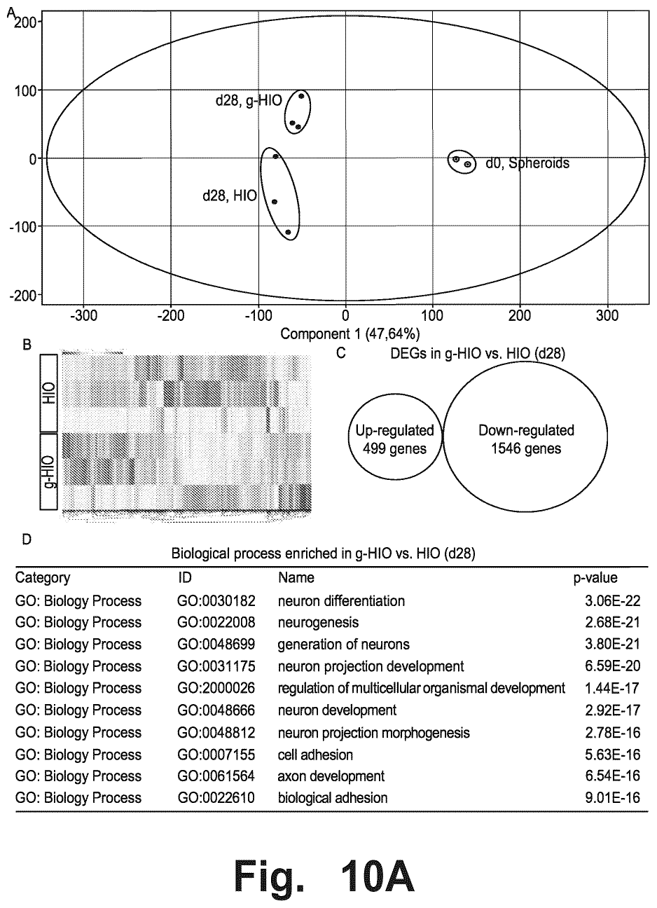

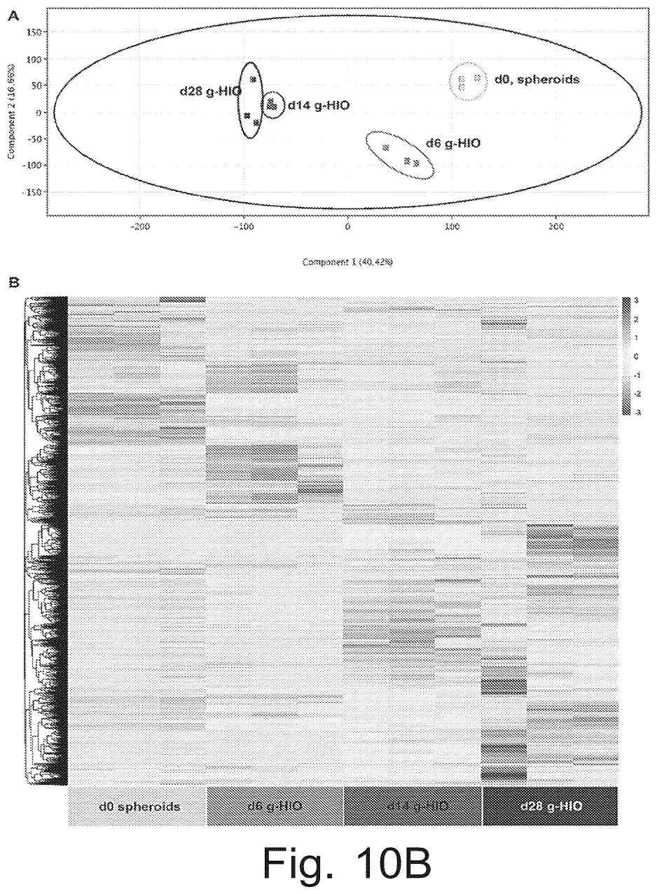

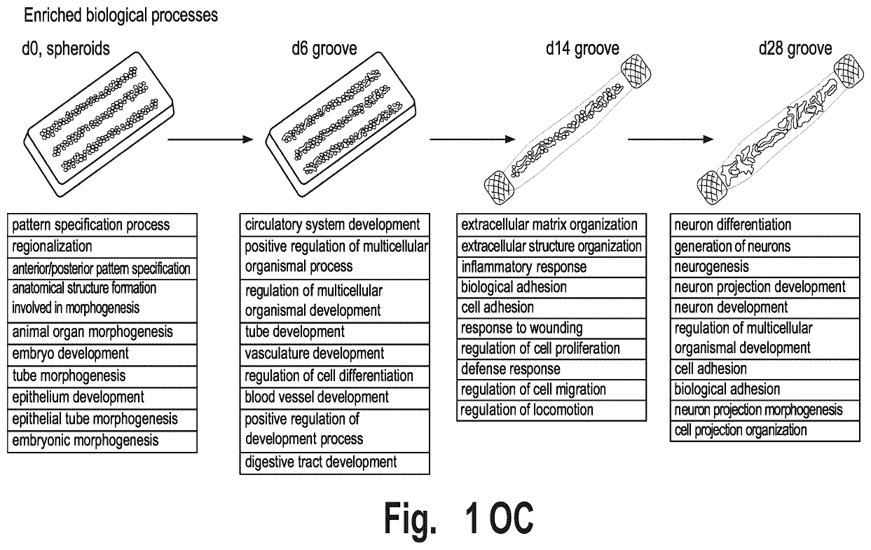

Gene expression profiles of mid-hindgut spheroids, unshaped intestinal organoids, and shaped elongated intestinal organoids were assessed. Day 28 culture g-HIOs exhibited 499 upregulated genes and 1546 downregulated genes relative to day 28 culture unshaped HIOs (FIG. 10A), demonstrating that the process of organoid shaping in a collection channel significantly alters biological activity in the constituent cells. Genes associated with neuron differentiation, neurogenesis, generation of neurons, neuron projection development, regulation of multicellular organismal development, neuron development, neuron projection morphogenesis, cell adhesion, axon development, and biological adhesion support the observation of spontaneous neurogenesis in the g-HIOs as well as development of the organoid into a mature, organ-tissue like state.

Gene expression profiles of g-HIOs grown in the collection channel at different culture times (day 0 [spheroids], day 6, day 14, and day 28) were also assessed (FIG. 10B). Each growth stage exhibited enrichment of genes associated with different biological processes (FIG. 10C). Day 0 spheroids were associated with pattern specification processes, regionalization, anterior/posterior pattern specification, anatomical structure formation involved in morphogenesis, animal organ morphogenesis, embryo development, tube morphogenesis, epithelium development, epithelial tube morphogenesis, and embryonic morphogenesis. Day 6 g-HIOs were associated with circulatory system development, positive regulation of multicellular organismal processes, regulation of multicellular organismal development, tube development, vasculature development, regulation of cell differentiation, blood vessel development, positive regulation of developmental processes, and digestive tract development. Day 14 g-HIOs were associated with extracellular matrix organization, extracellular structure organization, inflammatory response, biological adhesion, cell adhesion, response to wounding, regulation of cell proliferation, defense response, regulation of cell migration, and regulation of locomotion. Day 28 g-HIOs were associated with neuron differentiation, generation of neurons, neurogenesis, neuron projection development, neuron development, regulation of multicellular organismal development, cell adhesion, biological adhesion, neuron projection morphogenesis, and cell projection organization. These data suggest that significant developmental and morphological changes occur during the organoid culturing period, resembling the in vivo development of intestinal tissue.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

### REFERENCES

Selvaggi, G and Khan F. A. Overview of intestinal and multivisceral transplantation. UpToDate. 2019 November Available on the world wide web at uptodate.com/contents/overview-of-intestinal-and-multivisceral-transplantation/print
Abu-Elmagd et al. Current status of intestinal and multivisceral transplantation. Gastroentrerol Rep (Oxf). 2017 February; 5(1): 20-28
Ekser B, Kubal C A, Fridell J A, Mangus R S. Comparable outcomes in intestinal retransplantation: Single-center cohort study. Clin Transplant. 2018; 32:e13290.
Venick R S, Wozniak L J, Ngo K, et al. Unique technical and patient characteristics of retransplantation: a detailed single center analysis of intestinal transplantation. International Small Bowel Symposium 2013; Abstract 5.203 (Available on the world wide web at www.tts.org/component/tts/?view=presentation&id=13190)
Kubal, C A, Pennington K, Fridell J, Ekser B, Mihaylov P and Mangus R. Challenges with intestinal and multivisceral re-transplantation: importance of timing of re-transplantation and optimal immunosuppression. Ann Transplant, 2018; 23:98-104.
Hernandez F, Andres A M, Encinas J L, et al. Refining indications for intestinal retransplantation. International Small Bowel Symposium 2013; Abstract 12.241 (Available on the world wide web at www.tts.org/component/tts/?view=presentation&id=13241)
Loike, J D and Pollack, R. Opinion: Develop Organoids, Not Chimeras, for Transplantation. The Scientist Magazine. 2019 August https://www.the-scientist.com/news-opinion/opinion--develop-organoids--not-chimeras--for-transplantation-66339.
Wells et al. U.S. Pat. No. 9,719,068 B2. Aug. 1, 2017.
McCracken et al. Generating human intestinal tissue from pluripotent stem cells in vivo. Nat Protoc. 2011 Nov. 10; 6(12); 1920-1928.
A. Gurkan. Advances in small bowel transplantation. Turk J Surg. 2017; 33(3): 135-141.
McCracken et al. Generating human intestinal tissue from pluripotent stem cells in vivo. Nat Protoc. 2011 Nov. 10; 6(12); 1920-1928.
Cortez A R, Poling H M, Brown N E, Singh A, Mahe M M and Helmrath M A. Transplantation of Human Intestinal Organoids into the Mouse Mesentery: A More Physiologic and Anatomic Engraftment Site. Surgery. 2018 October; 164(4): 643-650.
Capeling et al. Nonadhesive alginate hydrogels support growth of pluripotent stem cell-derived intestinal organoids. Stem Cell Reports. 2019 February; (12): 381-394.
Wiley L A, Burnight E R, DeLuca A P, Anfinson K R, Cranston C M, Kaalberg E E, Penticoff J A, Affatigato L M, Mullins R F, Stone E M and Tucker B A. cGMP production of patient-specific iPSCs and photoreceptors precursor cells to treat retinal degenerative blindness. Scientific Reports 2016. DOI: 10.1038/srep30742.
Watson, C L et al. An in vivo model of human small intestine using pluripotent stem cells. Nat. Med. 2014. 20(11):1310-4.
Workman M J et al. Engineered human pluripotent-stem-cell-derived intestinal tissues with a functional enteric nervous system. Nat. Med. 2017. 23(1):49-59.
Poling, H M et al. Mechanically induced development and maturation of human intestinal organoids in vivo. Nat. Biomed. Eng. 2018. 2(6):429-442.
Mahe M M et al. In vivo model of small intestine. Methods Mol. Biol. 2017. 1597:229-245.
Grand, R J et al. Development of the human gastrointestinal tract. Gastroenterology. 1976. 80:790-810.

What is claimed is:

1. A shaped gastrointestinal organoid comprising a condensed mesenchyme and lumen, and wherein the shaped gastrointestinal organoid is non-spherical and comprises an elongated shape with a length and a width.

2. The shaped gastrointestinal organoid of claim 1, comprising an elongate length that is at least 1 mm, and a diameter that is at least 0.2 μm.

3. The shaped gastrointestinal organoid of claim 2, wherein the ratio of the elongate length to the diameter of the shaped gastrointestinal organoid is at least 2.

4. The shaped gastrointestinal organoid of claim 1, wherein the lumen is not continuous throughout the length of the shaped gastrointestinal organoid.

5. The shaped gastrointestinal organoid of claim 1, wherein the shaped gastrointestinal organoid is a shaped human gastrointestinal organoid.

6. The shaped gastrointestinal organoid of claim 1, further comprising enteric neuronal cells or enteric neuronal progenitor cells, and/or one or more myenteric plexuses comprising cells that express the neuronal marker PGP9.5.

7. The shaped gastrointestinal organoid of claim 1, wherein the shaped gastrointestinal organoid is innervated and/or has neuronal activity.

8. The shaped gastrointestinal organoid of claim 1, wherein the shaped gastrointestinal organoid is under mechanical strain.

9. The shaped gastrointestinal organoid of claim 8, wherein the mechanical strain is a uniaxial tensile strain.

10. The shaped gastrointestinal organoid of claim 1, further comprising a polarized, columnar epithelium surrounded by mesenchyme, wherein the mesenchyme comprises a smooth muscle-like layer.

11. The shaped gastrointestinal organoid of claim 1, further comprising an epithelium patterned into crypt-like proliferative zones or villus-like structures, or both.

12. The shaped gastrointestinal organoid of claim 1, further comprising laminated longitudinal and circular muscle.

13. The shaped gastrointestinal organoid of claim 1, further comprising markers of smooth muscle or intestinal sub-epithelial myofibroblast cells, or both.

14. The shaped gastrointestinal organoid of claim 1, further comprising one or more of enterocytes, enteroendocrine cells, goblet cells, Paneth cells, or any combination thereof.

15. The shaped gastrointestinal organoid of claim 1, further comprising cells that express one or more of villin, Muc2, DEFA5, CHGA, or OLFM4, or any combination thereof.

16. The shaped gastrointestinal organoid of claim 1, wherein the shaped gastrointestinal organoid is derived from induced pluripotent stem cells.

17. The shaped gastrointestinal organoid of claim 1, wherein the shaped gastrointestinal organoid is vascularized upon engraftment into an individual.

18. The shaped gastrointestinal organoid of claim 1, wherein the shaped gastrointestinal organoid is a shaped intestinal organoid, a shaped colonic organoid, a shaped esophageal organoid, or a shaped gastric organoid.

19. The shaped gastrointestinal organoid of claim 1, wherein the shaped gastrointestinal organoid is produced by a method comprising:
placing a plurality of spheroids into a confined predetermined shape in vitro; and
culturing the plurality of spheroids in vitro in the confined predetermined shape to differentiate the plurality of spheroids into the shaped gastrointestinal organoid having the predetermined shape.

20. The shaped gastrointestinal organoid of claim 19, wherein confined predetermined shape comprises a collection channel having a non-spherical and elongated shape, and wherein the confined predetermined shape comprises a length with opposing longitudinal sidewalls and a width with opposing lateral sidewalls.

21. The shaped gastrointestinal organoid of claim 19, wherein the plurality of spheroids is selected from the group consisting of:
a) a plurality of mid-hindgut spheroids, and wherein the shaped gastrointestinal organoid is a shaped intestinal organoid;
b) a plurality of hindgut spheroids, and wherein the shaped gastrointestinal organoid is a shaped colonic organoid;
c) a plurality of anterior foregut spheroids, and wherein the shaped gastrointestinal organoid is an esophageal organoid; and
d) a plurality of posterior foregut spheroids, and wherein the shaped gastrointestinal organoid is a gastric organoid.

22. The shaped gastrointestinal organoid of claim 19, wherein the spheroids are derived from induced pluripotent stem cells.
